(19) United States
(12) Patent Application Publication
(10) Pub. No.: US 2023/0233708 A1
(43) Pub. Date: Jul. 27, 2023

(54) ENHANCING AAV-MEDIATED DELIVERY AND TRANSDUCTION WITH POLYVINYL ALCOHOL

(71) Applicant: University of Florida Research Foundation, Incorporated, Gainesville, FL (US)

(72) Inventors: Keyun Qing, Gainesville, FL (US); Arun Srivastava, Gainesville, FL (US)

(73) Assignee: University of Florida Research Foundation, Incorporated, Gainesville, FL (US)

(21) Appl. No.: 17/918,887

(22) PCT Filed: Apr. 13, 2021
(86) PCT No.: PCT/US21/27169

§ 371 Date: Oct. 13, 2022

Related U.S. Application Data
(60) Provisional application No. 63/010,030, filed on Apr. 14, 2020.
(60) Provisional application No. 63/024,015, filed on May 13, 2020.

(51) Int. Cl. A61K 48/00

(52) U.S. Cl. CPC A61K 48/0041; A61K 48/0058

(57) ABSTRACT

Disclosed herein are novel methods for administration of rAAV particles having enhanced transduction properties, comprising the pre-incubation and co-administration of AAV capsids and polyvinyl alcohol (PVA). The disclosed methods of rAAV particle administration have improved efficiency in transducing mammalian liver and hematopoetic cells in vivo. The disclosed methods are suitable for use with a variety of AAV capsid serotypes and presudotypes and improve vector potency, thus lowering the concentration of rAAV particle required to achieve the desired effect. Further disclosed herein are buffers for storage and manufacturing of rAAV particles comprising PVA.

### CROSS-REFERENCE TO RELATED APPLICATIONS

This application claims the benefit of the filing dates of U.S. Provisional Application No. 63/010,030, filed Apr. 14, 2020, and U.S. Provisional Application No. 63/024,015, filed May 13, 2020, the entire contents of each of which are incorporated by reference.

### BACKGROUND OF THE INVENTION

Major advances in the field of gene therapy have been achieved by using viruses to deliver therapeutic genetic material. The adeno-associated virus (AAV) has attracted considerable attention as a highly effective viral vector for gene therapy due to its low immunogenicity and ability to effectively transduce non-dividing cells. AAV has been shown to infect a variety of cell and tissue types, and significant progress has been made over the last decade to adapt this viral system for use in human gene therapy. A handful of AAV therapies have recently been approved for use in patients by the FDA.

However, intrahepatic AAV therapies generally suffer from poor transduction profiles. Achieving adequate transduction of rAAV particles of the AAV6 serotype has been particularly elusive in part due to the propensity of AAV6 capsids to aggregate. This aggregation is in large part due to the presence of positively-charged amino acid residues on the capsid surface.

Gene therapy and genome editing of human hemoglobinopathies, such as β-thalassemia and sickle cell disease, is highly desirable because these are the most common human genetic diseases on earth. One out of every 600 humans globally suffers from one of these diseases. A permanent cure of sickle cell disease, the first molecularly defined human disease since 1957, is of particular significance because of the enormous physical and economic burdens worldwide. Furthermore, since the sickle mutation is among the most well-characterized, and all sickle cell patients have exactly the same mutation, the notion that if one patient can be cured, then thousands more patients can be cured, is enormously attractive.

Although gene therapy of hemoglobinopathies was attempted, at least in animal models, with recombinant retroviral vectors more than two decades ago [1], the low-levels of transgene expression, ranging from 0.04-0.56%, rendered this approach less desirable. However, there has been renewed interest in this pursuit for gene therapy of β-thalassemia and sickle-cell disease in recent years since the use of modified lentiviral vectors was first shown to lead to the production of potentially therapeutic levels of normal b-globin in homozygous b-thalassemic mice in vivo [2], and phenotypic correction of sickle-cell disease has also been achieved using modified lentiviral vectors [3]. Similar studies have been reported by a number of investigators [4-8]. The use of lentiviral vectors in clinical trials for both β-thalassemia and sickle cell disease have led to phenotypic correction of these diseases [9-11]. However, several studies have also reported the propensity of lentiviral vectors to integrate into active genes [12-15]. Genomic sequencing of vector containing fragments from CD34⁺ cells transduced with a lentiviral vector expressing anti-sickling β-globin showed that 86% of proviral integration occurred in genes [7]. Thus, there is a risk of insertional mutagenesis. Indeed, integration of a lentiviral vector leading to activation of a cellular proto-oncogene, HMGA2, in a patient with β-thalassemia has been reported [9]. Thus, the long-term safety of lentiviral vectors still remains to be determined.

### SUMMARY OF THE INVENTION

The present disclosure provides novel methods and buffers for administration of rAAV particles having enhanced transduction properties, comprising the pre-incubation and/or co-administration of AAV capsids and polyvinyl alcohol. These novel methods are safe, as they rely on treatment with a natural, non-toxic material. In addition, these methods do not rely on the modification of the capsid and function through a mechanism that does not interfere with AAV adhesion, thus fulfilling a long-felt need in the art. Aspects of the application are useful for enhancing transduction efficiencies of rAAV (e.g., rAAV6 and rAAV3) and are useful for use in therapy without using mutations in the AAV capsid and while avoiding treatment with non-toxic material.

Polyvinyl alcohol (PVA) is a water-soluble synthetic polymer consisting of an alkane backbone with hydroxyl groups attached at every second carbon. It thus consists of repeating CH₂CHOH units. It is an inexpensive high tonnage industrial chemical used in adhesives, textile manufacturing, food packaging, cosmetics, and pharmaceutical preparations. It is non-toxic, environmentally friendly, and biodegradable. PVA is typically manufactured by polymerization of vinyl acetate and subsequent hydrolysis of polyvinyl acetate. PVA is commercially available as partially hydrolyzed (some residual vinyl acetate units) or fully hydrolyzed (no residual vinyl acetate) polymers. PVA has been discussed as a potential vehicle for delivery of virus to bladder epithelial cells. See Ramesh et al., U.S. Patent Publication No. 2008/241105, herein incorporated by reference.

Advantageously, the novel methods of rAAV particle administration disclosed herein have improved efficiency in transducing liver cells and stem cells, and in particular, in transducing human liver and primary human stem cells in vivo.

Over the last decade, researchers have endeavored to improve the transduction efficiencies of rAAV particles in hepatic and other tissues. The bulk of these efforts have been focused on designing improved, “next generation” AAV having mutated capsid sequences that demonstrate enhanced transduction efficiencies when infecting targeted mammalian cells, such as hepatic cells. While the development of rAAV particles comprising capsid variants represents a promising area for advancement in the treatment of ocular diseases and disorders, fewer researchers have endeavored to enhance the transduction efficiencies of AAV by pre-treatment of capsids with exogenous material prior to administration to a subject. Prior methods designed to reduce aggregation of AAV6 capsids have involved pre-treatment of capsids with high-salt buffers. High-salt buffers may be toxic to hepatic tissues, however. The disclosed compositions and methods provide a solution to this toxicity problem and provide for improved transduction efficiencies of AAV6, AAV3 and other AAV serotypes. Improved transduction efficiencies may lead to lower doses of rAAV particles needed for therapy.

The pursuit of the potential gene therapy of β-thalassemia and sickle cell disease with adeno-associated virus (AAV) vectors [16] has long been an objective in the art. In these previous studies, while AAV2 vectors performed better (transgene expression ranging from 3-7%) than retroviral vectors, therapeutic levels of human β-globin gene expression could not be achieved in normal and b-thalassemic mice [16, 17] since AAV2 vectors do not efficiently transduce mouse hematopoietic stem cells (HSCs). In subsequent studies, identified AAV1 and AAV7 serotype vectors were identified as significantly more efficient than AAV2 vectors in transducing normal mouse HSCs, but these serotype vectors failed to transduce HSCs from sickle cell disease mice [18-20]. Thus, it was concluded that mouse models of HSC transduction and sickle cell disease are not a good surrogate, at least for AAV vectors. In 2013, it was first reported that the identification of AAV6 as the most efficient serotype for transduction of primary human HSCs [21]. Subsequently, three independent groups corroborated that AAV6 vectors are highly efficient in genome editing in primary human HSCs [22-24].

More recently, AAV6 vectors were reported to lead to successful genome editing of sickle mutation in primary human HSCs from patients with sickle cell disease [25]. However, multiplicities of infection (MOI) of 100,000-200,000 vgs/cell were required to achieve transduction efficiencies ranging between 45-55% in those studies, although different strategies have been explored to improve the transduction efficiency of AAV6vectors in human HSCs, such as the use (i) self-complimentary AAV vectors [26-28]; (ii) tropism specific promoters [29, 30]; and (iii) capsid-mutagenesis of AAV vectors [31-35].

The present disclosure provides methods that fulfill each of these needs in the art. The disclosure is based, at least in part, on the recent discovery that human serum albumin (HSA) was shown to increase the transduction efficiency of all AAV serotype vectors [36], and more recently, polyvinyl alcohol (PVA), was reported to be a superior replacement for HSA for ex vivo expansion of HSCs [37]. In the present disclosure, it was evaluated whether PVA could also enhance the transduction efficiency of AAV6 vectors in primary human HSCs. Experimental evidence is provided that PVA may increase the transduction efficiency of AAV6 vectors in primary human HSCs up to 12-fold, which is mediated through improved entry and intracellular trafficking of AAV6 vectors, both in vitro and in vivo. This has implications in the optimal use of these vectors in the potential gene therapy and genome editing for human hemoglobinopathies.

Advantageously, the disclosed methods are suitable for use with a variety of AAV capsid serotypes and presudotypes. The disclosed methods improve vector potency, thus lowering the concentration of rAAV particle required to achieve the desired effect. Accordingly, the disclosed methods are capable of increasing safety and reducing expense of manufacturing or rAAV. These methods minimize off-target effects of genome editing with AAV vectors, lower the cost of care, and increase the accessibility of gene therapies. The disclosed methods increase hepatic and stem cell transduction of AAV in any clinical setting where AAV6 or AAV3-based capsids are delivered by injection, e.g., intravenous injection. As provided in the present disclosure, pre-incubation of PVA with AAV6- and AAV3-based capsids resulted in more enhanced transduction efficiency than pre-incubation of PVA with AAV2-based capsids. AAV6- and AAV3-based capsids may have differential properties that confer improved compatibility with PVA or improved transduction effects for a PVA-capsid complex in cells, such as liver cells and HSCs. Likewise, AAVrh.74-capsids may have differential properties that confer improved compatibility with PVA or improved transduction effects for a PVA-capsid complex in cells, such as liver cells and HSCs.

Accordingly, in some aspects, the disclosure provides methods comprising co-administering an rAAV particle with polyvinyl alcohol by injection to one or both eyes of a mammal, wherein the rAAV particle comprises a capsid comprising one or more surface-exposed patches of positively-charged residues. In some embodiments, the capsid of the rAAV particle is pre-incubated with polyvinyl alcohol (PVA) prior to administration to the eyes of a mammal. In certain embodiments, the capsid is pre-incubated with a buffer that comprises PVA. In particular embodiments, the capsid is pre-incubated for a duration of 15 minutes.

In certain embodiments, the rAAV particles of the methods disclosed herein further comprises a polynucleotide comprising a heterologous nucleic acid sequence. The heterologous nucleic acid sequence may be operably linked to regulatory sequences which direct expression of the heterologous nucleic acid sequence in a hepatic cell.

In some embodiments, the rAAV particle is an rAAV3 or rAAV6 particle, or a variant thereof. Exemplary AAV3 and AAV6 variants are listed in Tables 2 and 3. As such, provided herein are methods for providing a mammal in need thereof with a therapeutically effective amount of a therapeutic agent, the methods comprising co-administering an rAAV particle with polyvinyl alcohol to one or both eyes of a mammal for a time effective to provide the mammal with a therapeutically-effective amount of the therapeutic agent, wherein the rAAV particle comprises an AAV6 or an AAV3 capsid. Likewise, further provided herein are methods for treating or ameliorating one or more symptoms of a disease, disorder or condition, comprising co-administering an rAAV particle with polyvinyl alcohol to one or both eyes of a mammal in need thereof for a time sufficient to treat or ameliorate the one or more symptoms of the disease, disorder or condition in the mammal, wherein the rAAV particle comprises i) a polynucleotide encoding a therapeutic agent and ii) an AAV3 or an AAV6 capsid. Mutagenic variants of AAV6 capsid are also contemplated for use in the compositions and methods of this disclosure. Mutagenic variants of AAV3 capsid are contemplated for use in the compositions and methods of this disclosure.

In some embodiments, the rAAV particle is an rAAVrh.74 (or AAVrh74) particle, or a variant thereof. Exemplary AAVrh74 variants are listed in Table 11. AAV rh.74 capsid protein exhibits about 93% identity to AAV8 capsid protein. As such, provided herein are methods for providing a mammal in need thereof with a therapeutically effective amount of a therapeutic agent, the methods comprising co-administering an rAAV particle with polyvinyl alcohol to one or both eyes of a mammal for a time effective to provide the mammal with a therapeutically-effective amount of the therapeutic agent, wherein the rAAV particle comprises an AAVrh.74 capsid. Likewise, further provided herein are methods for treating or ameliorating one or more symptoms of a disease, disorder or condition, comprising co-administering an rAAV particle with polyvinyl alcohol to one or both eyes of a mammal in need thereof for a time sufficient to treat or ameliorate the one or more symptoms of the disease, disorder or condition in the mammal, wherein the rAAV particle comprises i) a polynucleotide encoding a therapeutic agent and ii) an AAVrh.74 capsid. Mutagenic variants of AAVrh.74 capsid are also contemplated for use in the compositions and methods of this disclosure.

In some embodiments, the methods disclosed herein are provided for treatment of a mammal suffering from a disease, disorder or condition such as age-related macular degeneration (AMD), wet AMD, dry AMD, or geographic atrophy. In certain embodiments, the disease or disorder is retinitis pigmentosa or glaucoma. In particular embodiments, the mammal is human.

The co-administration methods disclosed herein are suitable for delivery of, and enhance the transduction capacity of, capsids that include AAV6, AAV3 and capsids derived from AAV6 and AAV3. These capsids also include AAVrh.74. These capsids also include AAV1, AAV5, AAV8 and AAV9.

In other aspects, the present disclosure provides buffers for storing a mixture of AAV and polyvinyl alcohol (PVA), comprising: HA; balanced salt solution (BSS); artificial cerebrospinal fluid; and phosphate buffered saline (PBS). In certain embodiments, the disclosed buffers PVA, balanced salt solution (BSS); artificial cerebrospinal fluid; Ringer's lactate solution; and TMN200 solution, and/or additional excipients.

### BRIEF DESCRIPTION OF THE DRAWINGS

The following drawings form part of the present specification and are included to demonstrate certain aspects of the present invention. The invention may be better understood by reference to the following description taken in conjunction with the accompanying drawings, in which like reference numerals identify like elements, and in which:

FIGS. 1A-1B show that PVA augments the transduction efficiency of AAV6 vectors in K562 cells in vitro. K562 cells were transduced with 1×10³ vgs/cell of scAAV6-CBAp-EGFP vectors with and without pre-incubation with PVA87 (FIG. 1A) or PVA99 (FIG. 1B) for at 4° C. for 2 hrs. Transgene expression was determined by flow cytometry 48 hrs post-transduction. Statistical significance is indicated as *P<0.05.

FIGS. 2A-2B show that the PVA-AAV6 interaction is a critical step in augmenting the transduction efficiency of AAV6 vectors. AAV6 vectors were pre-incubated with various indicated concentration of PVA87 and used to infect K562 cells at 3×10³ vgs/cell. As shown in FIG. 2A, transgene expression was determined by flow cytometry 48 hrs post-transduction as described for FIG. 1. The effect of PVA87 on AAV6 vector transduction was determined under various conditions as follows: (1) AAV6 alone, (2) pre-incubation of AAV6+1% PVA87, (3) pre-incubation of K562 cells with 1% PVA87, (4) addition of 1% PVA87 prior to AAV6 transduction, and (5) addition of 1% PVA87 2 h following transduction with AAV6 vectors (FIG. 2B). Transgene expression was determined by flow cytometry 48 hrs post-transduction as described above. Statistical significances are indicated as *P<0.05, **P<0.01, ***P<0.001.

FIGS. 3A-3B show that PVA improves AAV6 vector uptake in K562 cells in vitro. K562 cells were transduced with 1×10³ vgs/cell of scAAV-CBAp-EGFP vectors, with or without pre-incubation with various indicated amounts of PVA87. Low molecular weight DNA samples were isolated 2 hrs post-transductions, and analyzed on a Southern blot using 32P-labeled EGFP-specific DNA probe (FIG. 3A). Quantitation of the data using the ImageX software (FIG. 3B).

FIG. 4 shows that PVA improves nuclear transport of AAV6 vectors in K562 cells in vitro. Southern blot analysis was performed with low mol. DNA samples isolated from cytoplasmic and nuclear fractions from K562 cells transduced with 3×10³ vgs/cell of scAAV6-CBAp-EGFP vectors, with and without pre-incubation with various concentration of PVA87, and probed with the EGFP-specific probed as described above (left panel). Densitometric scanning of the Southern blot and quantitation of the data were performed using the ImageX software for cytoplasmic (Cyto) (top right), and nuclear (Nuc) (bottom right) fractions.

FIGS. 5A-5B show that PVA increases the transduction efficiency of AAV6 vectors in primary human HSCs in vitro. Primary human bone marrow-derived CD34+ cells were transduced with scAAV6-CBAp-EGFP vectors at 3×10³ (FIG. 5A) and 1×10⁴ vgs/cell (FIG. 5B), with or without pre-incubation with 1% or 3% PVA87 at 4° C. for 2 hrs. Transgene expression was determined by flow cytometry 48 hrs post-transduction. Statistical significances are indicated as **P<0.01, ***P<0.001.

FIGS. 6A-6C show that PVA augments the transduction efficiency of AAV6 vectors in mouse liver in vivo. ssAAV6-CBAp-FLuc vectors, with or without pre-incubation with 1% or 3% PVA87 at 4° C. for 2 hrs, were injected via the tail vein in C57BL/6 mice at 1×10¹⁰ vgs/mouse. Whole-body bioluminescence images were acquired 2-weeks post-vector administration (FIG. 6A). Quantitation of bioluminescence signal intensity is shown as photons/second/cm2/steradian (p/sec/cm2/sr) (FIG. 6B). AAV6 vector genome copy numbers in mouse liver were quantified by qPCR using FLuc-specific primers (FIG. 6C). Statistical differences are indicated as *P<0.05, **P<0.01. RLU, relative light units; gDNA, genomic DNA.

FIGS. 7A-7B show that PVA-treatment is non-cytotoxic and does not affect the viability of human K562 cells. Flow cytometric profiles and scatter plots for mock-transduced, or AAV6 vector-transduced K562 cells, with and without pre-incubation with various concentrations of PVA87 (FIG. 7A) and PVA99 (FIG. 7B), are shown.

FIGS. 8A-8B show that higher concentrations of PVA under various conditions does not lead to cytotoxicity in human K562 cells. Flow cytometric profiles and scatter plots for mock-transduced, or AAV6 vector-transduced K562 cells, with and without pre-incubation with various concentrations of PVA87 (FIG. 8A), under various indicated conditions (FIG. 8B), are shown.

FIG. 9 shows that PVA-treatment is non-cytotoxic to primary human hematopoietic stem cells. Flow cytometric profiles and scatter plots for mock-transduced, or AAV6 vector-transduced primary human CD34+ cells at 3,000 vgs/cell, or 10,000 vgs/cell, with and without pre-incubation with various indicated concentrations of PVA87, are shown.

### DEFINITIONS

Unless defined otherwise, all technical and scientific terms used herein have the same meaning as commonly understood by one of ordinary skill in the art to which this disclosure belongs. Although any methods and compositions similar or equivalent to those described herein can be used in the practice or testing of the present disclosure, the preferred methods and compositions are described herein. For purposes of the present disclosure, the following terms are defined below:

As used herein, the terms “polyvinyl alcohol” and “PVA” encompass polyvinyl alcohol, without regard to the molecular weight or mass thereof. That is, these terms are meant to encompass polyvinyl alcohols of any molecular weight known or used in the art. In particular embodiments, the PVA of the disclosed compositions and methods encompasses PVA having an average molecular weight of about 30,000, 40,000, 50,000, 60,000, 70,000, 80,000, 85,000, 90,000, 100,000, 110,000, 120,000 or 125,000. In some embodiments, the PVA has an average molecular weight range of 30,000-70,000. In other embodiments, the PVA has an average molecular weight range of 85,000-124,000. Average molecular weight of PVA may be measured by any technique known in the art, such as viscosity measurements, rheological measurements, mass spectroscopy, polydispersity, static laser scattering, and size exclusion chromatography. In particular, viscosity measurements may be used. Average molecular weight may be expressed as number-average molecular weight (Mn) or mass-average molecular weight (Mw). These terms are meant to encompass commercial grade and non-commercial grade PVA. These terms also encompass variants of polyvinyl alcohol, including but not limited to truncated and chemically modified versions of PVA. A “variant” of PVA is at least about 80% identical, at least about 90% identical, at least about 95% identical, at least about 96% identical, at least about 97% identical, at least about 98% identical, at least about 99% identical, at least about 99.5% identical, or at least about 99.9% identical to a wild type PVA sequence.

The term “subject,” as used herein, describes an organism, including mammals such as primates, to which treatment with the compositions according to the present disclosure can be provided. Mammalian species that can benefit from the disclosed methods of treatment include, but are not limited to, apes; chimpanzees; orangutans; humans; monkeys; domesticated animals such as dogs and cats; livestock such as horses, cattle, pigs, sheep, goats, and chickens; and other animals such as mice, rats, guinea pigs, and PVAmsters.

The term “treatment” or any grammatical variation thereof (e.g., treat, treating, and treatment etc.), as used herein, includes but is not limited to, alleviating a symptom of a disease or condition; and/or reducing, suppressing, inhibiting, lessening, ameliorating or affecting the progression, severity, and/or scope of a disease or condition.

As used herein, the term “pre-treatment” refers to the application of exogenous material to an AAV vector, prior to administration of the vector to a subject or subject cell.

The term “effective amount,” as used herein, refers to an amount that is capable of treating or ameliorating a disease or condition or otherwise capable of producing an intended therapeutic effect.

As used herein, the terms “engineered” and “recombinant” cells are intended to refer to a cell into which an exogenous polynucleotide segment (such as DNA segment that leads to the transcription of a biologically active molecule) has been introduced. Therefore, engineered cells are distinguishable from naturally occurring cells, which do not contain a recombinantly introduced exogenous DNA segment. Engineered cells are, therefore, cells that comprise at least one or more heterologous nucleic acid segments introduced through the hand of man.

The term “promoter,” as used herein refers to a region or regions of a nucleic acid sequence that regulates transcription.

The term “regulatory element,” as used herein, refers to a region or regions of a nucleic acid sequence that regulates transcription. Exemplary regulatory elements include, but are not limited to, enhancers, post-transcriptional elements, transcriptional control sequences, and such like.

The term “vector,” as used herein, refers to a nucleic acid molecule (typically comprised of DNA) capable of replication in a host cell and/or to which another nucleic acid segment can be operatively linked so as to bring about replication of the attached segment. A plasmid, cosmid, or a virus is an exemplary vector.

The term “substantially corresponds to,” “substantially homologous,” or “substantial identity,” as used herein, denote a characteristic of a nucleic acid or an amino acid sequence, wherein a selected nucleic acid or amino acid sequence has at least about 70 or about 75 percent sequence identity as compared to a selected reference nucleic acid or amino acid sequence. More typically, the selected sequence and the reference sequence will have at least about 76, 77, 78, 79, 80, 81, 82, 83, 84 or even 85 percent sequence identity, and more preferably, at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared. The percentage of sequence identity may be calculated over the entire length of the sequences to be compared, or may be calculated by excluding small deletions or additions which total less than about 25 percent or so of the chosen reference sequence. The reference sequence may be a subset of a larger sequence, such as a portion of a gene or flanking sequence, or a repetitive portion of a chromosome. However, in the case of sequence homology of two or more polynucleotide sequences, the reference sequence will typically comprise at least about 18-25 nucleotides, more typically at least about 26 to 35 nucleotides, and even more typically at least about 40, 50, 60, 70, 80, 90, or even 100 or so nucleotides.

When highly-homologous fragments are desired, the extent of percent identity between the two sequences will be at least about 80%, preferably at least about 85%, and more preferably about 90% or 95% or higher, as readily determined by one or more of the sequence comparison algorithms well-known to those of skill in the art, such as e.g., the FASTA program analysis described by Pearson and Lipman (1988).

The term “operably linked,” as used herein, refers to that the nucleic acid sequences being linked are typically contiguous, or substantially contiguous, and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

As used herein, the term “variant” refers to a molecule (e.g. a capsid) having characteristics that deviate from what occurs in nature, e.g., a “variant” is at least about 80% identical, at least about 90% identical, at least about 95% identical, at least about 96% identical, at least about 97% identical, at least about 98% identical, at least about 99% identical, at least about 99.5% identical, or at least about 99.9% identical to the wild type capsid. Variants of a protein molecule, e.g. a capsid, may contain modifications to the amino acid sequence (e.g., having 1, 2, 3, 4, 5, 6, 7, 8, 9, 10, 10-15, or 15-20 amino acid substitutions) relative to the wild type protein sequence, which arise from point mutations installed into the nucleic acid sequence encoding the capsid protein. These modifications include chemical modifications as well as truncations.

### DETAILED DESCRIPTION OF THE INVENTION

Illustrative embodiments of the disclosure are described below. The present disclosure provides methods of use of PVA as a vehicle for administration of AAV particles to the eyes of a mammal via injection. The presently disclosed methods provide for pre-incubation with PVA and/or co-administration with PVA. The disclosure further provides buffers for storage of PVA and AAV capsids.

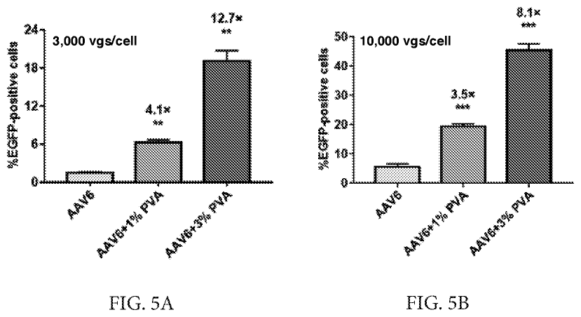

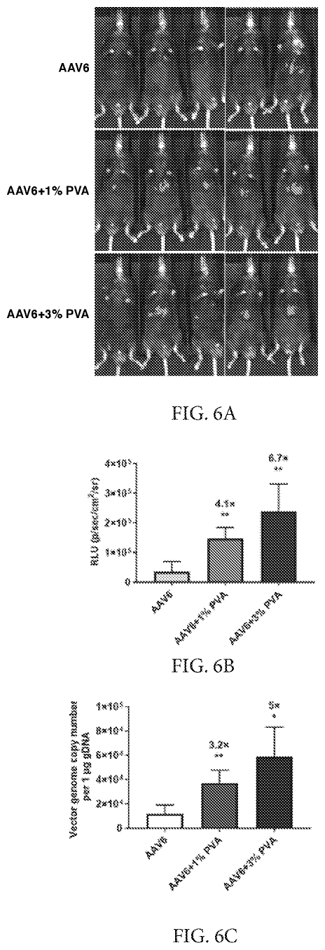

Out of the 10 most commonly used adeno-associated virus (AAV) serotype vectors, AAV6 is the most efficient in transducing primary human hematopoietic stem cells (HSCs) in vitro as well as in vivo (Cytother., 15, 986-998; 2013; PLoS One, 8: e58757, 2013; Sci. Rep., 2016. 6: p. 35495, 2013). More recently, polyvinyl alcohol (PVA), was reported to be a superior replacement for human serum albumin (HSA) for ex vivo expansion of HSCs (Nature, 571: 117-121, 2019). Since HSA has been shown to increase the transduction efficiency of AAV serotype vectors in general (Gene Ther., 24: 49-59, 2017), experiments were performed to evaluate the effect of PVA on transduction efficiency of AAV6 vectors in human hematopoietic cell line, K562. A dose-dependent increase of up to 9-fold was observed. Without wishing to be bound by theory, it is believe that the improvement in the transduction efficiency is due to PVA-mediated improved entry and intracellular trafficking of AAV6 vectors in human hematopoietic cells. PVA also was found to mediate up to 12-fold enhancement in the transduction efficiency of AAV6 vectors in primary human HSCs cells in vitro (FIG. 5) as well as up to 7-fold increase in murine hepatocytes in vivo (FIG. 6).

Taken together, the present disclosure provides that the use of PVA may further improve the efficacy of AAV6 and AAV3 vectors, which has important implications for effective use of these vectors in gene therapy and genome editing for human diseases that can be targeted using rAAV therapy (e.g., gene therapy delivered using rAAV6 vectors and rAAV3 vectors), for example hemoglobinopathies such as beta-thalassemia and sickle cell disease. The present disclosure provides that the use of PVA may further improve the efficacy of AAVrh.74 vectors.

As demonstrated in the below experiments, pre-incubation of PVA with AAV6- and AAV3-based capsids resulted in more enhanced transduction efficiency than pre-incubation of PVA with AAV2-based capsids. AAV6- and AAV3-based capsids may have differential properties that confer improved compatibility with PVA or improved transduction effects for a PVA-capsid complex in cells, such as liver cells and HSCs. Cryo-EM studies are currently being pursued with AAV2, AAV3, and AAV6 vectors to determine the capsid site(s) with which PVA interacts. The experiments of this disclosure may better elucidate the mechanism of transduction efficiency of AAV capsids by PVA.

In some embodiments of the methods disclosed herein, the capsid is pre-incubated with the PVA for a duration of about 5 minutes, about 15 minutes, about 30 minutes, about 45 minutes, about 60 minutes, about 75 minutes, about 90 minutes, or about 180 minutes prior to administration to a subject. In particular embodiments, the capsid is pre-incubated with PVA for about 15 minutes. In particular embodiments, the capsid is pre-incubated with PVA for more than about 15 minutes. In some embodiments, shorter incubation times may be used. In some embodiments, longer incubation times may be used.

In various embodiments of the disclosed methods, empty capsid that has not yet been packaged with rAAV vector is pre-treated, pre-contacted, or pre-incubated with the PVA. In other embodiments, the capsid is not pre-incubated with PVA prior to packaging with the rAAV vector, but rather the capsid is contacted with PVA at about the same time as, simultaneously with, or immediately after the capsid is contacted with rAAV vector during packaging. In some embodiments, PVA is added with one or more additional reagents. In some embodiments, PVA is added alone, in the absence of additional reagents.

In particular embodiments, the capsid is pre-incubated with buffer comprising PVA about 15 minutes, or more than about 15 minutes. In certain embodiments, the capsid is pre-incubated with buffer comprising PVA in a concentration of 0.01%, 0.05%, 0.1%, 0.2%, 0.3%, 0.4%, 0.5%, 0.6%, 0.75%, 0.9%, 1.0%, 1.25%, 1.5%, 1.75%, 2.0%, 2.25%, 2.5%, 2.75%, 3.0%, 3.25%, 3.5%, 3.75%, 4.0%, 5.0%, or 6.0% weight by volume (w/v) and BSS. In still further embodiments, the capsid is pre-incubated with buffer comprising PVA in a concentration of about 1.0% w/v, and one or more of BSS, artificial cerebrospinal fluid (CSF), and PBS. In some embodiments, concentrations lower than 1.0% w/v may be used. In some embodiments, higher concentrations may be used.

In some embodiments of the disclosed methods, the capsid is pre-incubated (or co-administered) with PVA in a concentration of 0.01%, 0.05%, 0.1%, 0.2%, 0.3%, 0.4%, 0.5%, 0.6%, 0.75%, 0.9%, 1.0%, 1.25%, 1.5%, 1.75%, 2.0%, 2.25%, 2.5%, 2.75%, 3.0%, 3.25%, 3.5%, 3.75%, 4.0%, 5.0%, or 6.0% w/v. In particular embodiments, the capsid is pre-incubated with PVA in a concentration of about 1.0% w/v. In other embodiments, the capsid is pre-incubated with PVA in a concentration of about 3.0% w/v. In some embodiments, concentrations lower than 1.0% w/v may be used. In some embodiments, higher concentrations may be used. In some embodiments, the capsid is pre-incubated or co-administered with hydrolyzed PVA in a concentration other than about 0.4%.

In some embodiments of the disclosed methods, the the capsid is pre-incubated with PVA that is hydrolyzed to about 80%, 82%, 83% 85%, 87%, 89%, 91%, 93%, 95%, 97%, 99%, or more than 99%. In certain embodiments, the capsid is pre-incubated with 87% hydrolyzed PVA. In some embodiments, the capsid is pre-incubated with 83% hydrolyzed PVA. In other embodiments, the capsid is pre-incubated with 99% hydrolyzed PVA.

In some embodiments, the rAAV particle is administered via injection in a titer of about 1×10⁶ vg/ml 1×10⁷ vg/ml, 1×10⁸ vg/ml, 5×10⁸ vg/ml, 1×10⁹ vg/ml, 5×10⁹ vg/ml, 1×10¹° vg/ml, 5×10¹⁰ vg/ml, 1×10¹¹ vg/ml, 5×10¹¹ vg/ml, 1×10¹² vg/ml, 2×10¹² vg/ml, 3×10¹² vg/ml, 4×10¹² vg/ml, 5×10¹² vg/ml, 1×10¹³ vg/ml, or 5×10¹³ vg/ml. In some embodiments, the rAAV particle is administered in a titer of less than 5×10¹² vg/ml. In some embodiments, the rAAV particle is administered in a titer of less than 5×10¹⁰ vg/ml. In particular embodiments, the rAAV particle is administered in a titer of less than 1×10⁹ vg/ml. In particular embodiments, the rAAV particle is administered in a titer of less than 5×10⁸ vg/ml. The lower end of these titers represents substantially lower doses than those doses routinely used in intravenous AAV delivery. In some embodiments, higher titers may be used.

In the methods disclosed herein, a mixture of rAAV and PVA is administered in an intravenous injection. In some embodiments, the intravenous injection is provided in a volume of about 500 μL, about 300 μL, about 250 μL, about 200 μL, about 175 μL, about 160 μL, about 145 μL, about 130 μL, about 115 μL, about 100 μL, about 90 μL, about 80 μL, about 70 μL, about 60 μL, about 55 μL, about 50 μL, about 45 μL, about 35 μL, about 20 μL, about 10 μL, or about 5 μL. In particular embodiments, the intravenous injection is provided in a volume of about 200 μL. In particular embodiments, the intravenous injection is provided in a volume of about 50 μL. In some embodiments, lower volumes may be used. In some embodiments, higher volumes may be used.

In certain aspects of the disclosed methods, higher concentrations of AAV vector can be delivered in a given volume of administration by intravenous injection.

In other aspects of the disclosure, a method for providing a mammal in need thereof with a therapeutically-effective amount of a selected therapeutic agent is disclosed herein. In embodiments of the invention, the therapeutic agent is encoded in a heterologous nucleic acid, or transgene, that is inserted into a recombinant AAV nucleic acid vector. Such a method generally includes at least the step of administering to one or both eyes of the mammal, an amount of one or more of the rAAV particles disclosed herein; for a time effective to provide the mammal with a therapeutically-effective amount of the selected therapeutic agent.

The method may include, for example, the step of administering (a single time or multiple times) to the liver cells of the mammal, an amount of one or more rAAV particles disclosed herein; and for a time effective to provide the mammal with a therapeutically-effective amount of the selected diagnostic or therapeutic agent.

In some embodiments, the therapeutic agent is a therapeutic protein. In certain embodiments, the therapeutic protein is an enzyme, a hormone, an antibody, a receptor (e.g., a cell surface receptor), or other protein than can useful in therapy. In some embodiments, a protein is a human protein. In some embodiments, a protein is from a mammal other than a human. In particular embodiments, the therapeutic protein is selected from a clotting factor (e.g., Factor VIII and Factor IX) or a globin (e.g., human β-globin and human γ-globin). In some embodiments, the therapeutic protein is Factor VIII. In some embodiments, the therapeutic protein is Factor IX (e.g., wild-type or Padua mutant). In some embodiments, the therapeutic protein is human β-globin. In some embodiments, the therapeutic protein is human γ-globin.

In some embodiments, the nucleic acid vector comprises one or more transgenes comprising a sequence encoding a protein or polypeptide of interest, such as a therapeutic protein provided in Table 1 or described herein.

The transgene encoding the protein or polypeptide of interest may be, e.g., a polypeptide or protein of interest provided in Table 1. The sequences of the polypeptide or protein of interest may be obtained, e.g., using the non-limiting National Center for Biotechnology Information (NCBI) Protein IDs or SEQ ID NOs from patent applications provided in Table 1.

TABLE 1
Non-limiting examples of proteins or polypeptides of interest and associated diseases

| | | Exemplary NCBI Protei [symbol] |
|---|---|---|
| | Non-limiting Exemplary | IDs or Patent SEQ ID |
| Protein or Polypeptide | diseases | NOs |
| acid alpha-glucosidase (GAA) | Pompe Disease | NP_000143.2, |
| | | NP_001073271.1, |
| | | NP_001073272.1 |
| Methyl CpG binding protein 2 | Rett syndrome | NP_001104262.1, |
| (MECP2) | | NP_004983.1 |
| Aromatic L-amino acid | Parkinson's disease | NP_000781.1, |
| decarboxylase (AADC) | | NP_001076440.1, |
| | | NP_001229815.1, |
| | | NP_001229816.1, |
| | | NP_001229817.1, |
| | | NP_001229818.1, |
| | | NP_001229819.1 |
| Glial cell-derived neurotrophic | Parkinson's disease | NP_000505.1, |
| factor (GDNF) | | NP_001177397.1, |
| | | NP_001177398.1, |
| | | NP_001265027.1, |
| | | NP_954701.1 |
| Cystic fibrosis transmembrane | Cystic fibrosis | NP_000483.3 |
| conductance regulator (CFTR) | | |
| Tumor necrosis factor receptor | Arthritis, Rheumatoid arthriti [symbol] | SEQ ID NO: 1 of WO |
| fused to an antibody Fc (TNFR:Fc [symbol] | | 2013/025079 |
| HIV-1 gag-proΔrt (tgAAC09) | HIV infection | SEQ ID NOs: 1-5 of WO |
| | | 2006/073496 |
| Sarcoglycan alpha, beta, gamma, | Muscular dystrophy | SGCA |
| delta, epsilon, or zeta (SGCA, | | NP_000014.1, |
| SGCB, SGCG, SGCD, SGCE, or | | NP_001129169.1 |
| SGCZ) | | SGCB |
| | | NP_000223.1 |
| | | SGCG |
| | | NP_000222.1 |
| | | SGCD |
| | | NP_000328.2, |
| | | NP_001121681.1, |
| | | NP_758447.1 |
| | | SGCE |
| | | NP_001092870.1, |
| | | NP_001092871.1, |
| | | NP_003910.1 |
| | | SGCZ |
| | | NP_631906.2 |
| Alpha-1-antitrypsin (AAT) | Hereditary emphysema or | NP_000286.3, |
| | Alpha-1-antitrypsin | NP_001002235.1, |
| | Deficiency | NP_001002236.1, |
| | | NP_001121172.1, |
| | | NP_001121173.1, |
| | | NP_001121174.1, |
| | | NP_001121175.1, |
| | | NP_001121176.1, |
| | | NP_001121177.1, |
| | | NP_001121178.1, |
| | | NP_001121179.1 |
| Glutamate decarboxylase | Parkinson's disease | NP_000808.2, |
| 1(GAD1) | | NP_038473.2 |
| Glutamate decarboxylase | Parkinson's disease | NP_000809.1, |
| 2 (GAD2) | | NP_001127838.1 |
| Aspartoacylase (ASPA) | Canavan's disease | NP_000040.1, |
| | | NP_001121557.1 |
| Nerve growth factor (NGF) | Alzheimer's disease | NP_002497.2 |
| Granulocyte-macrophage | Prostate cancer | NP_000749.2 |
| colonystimulating | | |
| factory (GM-CSF) | | |
| Cluster of Differentiation 86 | Malignant melanoma | NP_001193853.1, |
| (CD8 [symbol] or B7-2) | | NP_001193854.1, |
| | | NP_008820.3, |
| | | NP_787058.4, |
| | | NP_795711.1 |
| Interleukin 12 (IL-12) | Malignant melanoma | NP_000873.2, |
| | | NP_002178.2 |
| neuropeptide Y (NPY) | Parkinson's disease, epilepsy | NP_000896.1 |
| ATPase, Ca++ transporting, cardia [symbol] | Chronic heart failure | NP_001672.1, |
| muscle, slow twitch 2 (SERCA2) | | NP_733765.1 |
| Dystrophin or Minidystrophin | Muscular dystrophy | NP_000100.2, |
| | | NP_003997.1, |
| | | NP_004000.1, |
| | | NP_004001.1, |
| | | NP_004002.2, |
| | | NP_004003.1, |
| | | NP_004004.1, |
| | | NP_004005.1, |
| | | NP_004006.1, |
| | | NP_004007.1, |
| | | NP_004008.1, |
| | | NP_004009.1, |
| | | NP_004010.1, |
| | | NP_004011.2, |
| | | NP_004012.1, |
| | | NP_004013.1, |
| | | NP_004014.1 |
| Ceroid lipofuscinosis neuronal 2 | Late infantile neuronal | NP_000382.3 |
| (CLN2) | ceroidlipofuscinosis or | |
| | Batten's disease | |
| Neurturin (NRTN) | Parkinson's disease | NP_004549.1 |
| N-acetylglucosaminidase, alpha | Sanfilippo syndrome | NP_000254.2 |
| (NAGLU) | (MPSIIIB) | |
| Iduronidase, alpha-1 (IDUA) | MPSI-Hurler | NP_000194.2 |
| Iduronate 2-sulfatase (IDS) | MPSII-Hunter | NP_000193.1, |
| | | NP_001160022.1, |
| | | NP_006114.1 |
| Glucuronidase, beta (GUSB) | MPSVII-Sly | NP_000172.2, |
| | | NP_001271219.1 |
| Hexosaminidase A, α polypeptide | Tay-Sachs | NP_000511.2 |
| (HEXA) | | |
| Retinal pigment epithelium- | Leber congenital amaurosis | NP_000320.1 |
| specifi [symbol] protein 65 kDa (RPE65) | | |
| Factor IX (FIX) | Hemophilia B | NP_000124.1 |
| Factor IX, Padua mutant | | |
| Adenine nucleotide translocator | progressive external | NP_001142.2 |
| (ANT-1) | ophthalmoplegia | |
| ApaLI | mitochondrial heteroplasmy, | YP_007161330.1 |
| | myoclonic epilepsy with | |
| | ragged | |
| | red fibers (MERRF) or | |
| | mitochondrial | |
| | encephalomyopathy, | |
| | lactic acidosis, and stroke- | |
| | lik [symbol] episodes (MELAS) | |
| NADH ubiquinone | Leber hereditary | YP_003024035.1 |
| oxidoreductase subunit 4 (ND4) | Optic | |
| very long-acyl-CoA | very long-chain acyl-CoA | NP_000009.1, |
| dehydrogenas [symbol] | dehydrogenase (VLCAD) | NP_001029031.1, |
| (VLCAD) | deficiency | NP_001257376.1, |
| | | NP_001257377.1 |
| short-chain acyl-CoA | short-chain acyl-CoA | NP_000008.1 |
| dehydrogenase (SCAD) | dehydrogenase (SCAD) | |
| | deficiency | |
| medium-chain acyl-CoA | medium-chain acyl-CoA | NP_000007.1, |
| dehydrogenase (MCAD) | dehydrogenase (MCAD) | NP_001120800.1, |
| | deficiency | NP_001272971.1, |
| | | NP_001272972.1, |
| | | NP_001272973.1 |
| Myotubularin 1 (MTM1) | X-linked myotubular | NP_000243.1 |
| | myopathy | |
| Myophosphorylase (PYGM) | McArdle disease (glycogen | NP_001158188.1, |
| | storage disease type V, | NP_005600.1 |
| | myophosphorylase | |
| | deficiency) | |
| Lipoprotein lipase (LPL) | LPL deficiency | NP_000228.1 |
| sFLT01 (VEGF/PlGF (placental | Age-related macular | SEQ ID NOs: 2, 8, 21, |
| growth factor) binding domain of | degeneration | 23 [symbol] or 25 of WO |
| human VEGFR1/Flt-1 (hVEGFR1 | | 2009/10566 [symbol] |
| fused to the Fc portion of human | | |
| IgG(1) through a polyglycine | | |
| linker) | | |
| Glucocerebrosidase (GC) | Gaucher disease | NP_000148.2, |
| | | NP_001005741.1, |
| | | NP_001005742.1, |
| | | NP_001165282.1, |
| | | NP_001165283.1 |
| UDP glucuronosyltransferase 1 | Crigler-Najjar syndrome | NP_000454.1 |
| family, polypeptide Al (UGT1A1) | | |
| Glucose 6-phosphatase (G6Pase) | GSD-Ia | NP_000142.2, |
| | | NP_001257326.1 |
| Ornithine carbamoyltransferase | OTC deficiency | NP_000522.3 |
| (OTC) | | |
| Cystathionine-beta-synthase (CBS [symbol] | Homocystinuria | NP_000062.1, |
| | | NP_001171479.1, |
| | | NP_001171480.1 |
| Factor VIII (F8) | Hemophilia A | NP_000123.1, |
| | | NP_063916.1 |
| Hemochromatosis (HFE) | Hemochromatosis | NP_000401.1, |
| | | NP_620572.1, |
| | | NP_620573.1, |
| | | NP_620575.1, |
| | | NP_620576.1, |
| | | NP_620577.1, |
| | | NP_620578.1, |
| | | NP_620579.1, |
| | | NP_620580.1 |
| Low density lipoprotein receptor | Phenylketonuria (PKU) | NP_000518.1, |
| (LDLR) | | NP_001182727.1, |
| | | NP_001182728.1, |
| | | NP_001182729.1, |
| | | NP_001182732.1 |
| Galactosidase, alpha (AGA) | Fabry disease | NP_000160.1 |
| Phenylalanine | Hypercholesterolaemia or | NP_000268.1 |
| hydroxylase (PAH) | Phenylketonuria (PKU) | |
| Propionyl CoA carboxylase, alpha | Propionic acidaemias | NP_000273.2, |
| polypeptide (PCCA) | | NP_001121164.1, |
| | | NP_001171475.1 |

[symbol]

indicates data missing or illegible when filed

Other exemplary polypeptides or proteins of interest include clotting factors (e.g., Factor VIII and Factor IX), globins (e.g., human β-globin and human γ-globin), adrenergic agonists, anti-apoptosis factors, apoptosis inhibitors, cytokine receptors, cytokines, cytotoxins, erythropoietic agents, glutamic acid decarboxylases, glycoproteins, growth factors, growth factor receptors, hormones, hormone receptors, interferons, interleukins, interleukin receptors, kinases, kinase inhibitors, nerve growth factors, netrins, neuroactive peptides, neuroactive peptide receptors, neurogenic factors, neurogenic factor receptors, neuropilins, neurotrophic factors, neurotrophins, neurotrophin receptors, N-methyl-D-aspartate antagonists, plexins, proteases, protease inhibitors, protein decarboxylases, protein kinases, protein kinsase inhibitors, proteolytic proteins, proteolytic protein inhibitors, semaphoring, semaphorin receptors, serotonin transport proteins, serotonin uptake inhibitors, serotonin receptors, serpins, serpin receptors, and tumor suppressors. In some embodiments, the polypeptide or protein of interest is a human protein or polypeptide.

In other aspects, the disclosure provides a method for treating or ameliorating one or more symptoms of a disease, a disorder, a dysfunction, an injury, an abnormal condition, or trauma in a mammal. In an overall and general sense, such a method includes at least the step of administering to one or both eyes of the mammal in need thereof, one or more of the disclosed rAAV particles herein, in an amount and for a time sufficient to treat or ameliorate the one or more symptoms of the disease, the disorder, the dysfunction, the injury, the abnormal condition, or the trauma in the mammal.

In another embodiment, the disclosure provides a method for expressing a heterologous nucleic acid segment in one or more erythroid cells or one or more hepatic cells of a mammal (e.g., a human). In an overall and general sense, such a method includes administering (e.g., directly administering intravenously) to the mammal one or more of the rAAV particles disclosed herein, wherein the polynucleotide further comprises at least a first polynucleotide that comprises a hepatocyte-specific promoter operably linked to at least a first heterologous nucleic acid segment that encodes a therapeutic agent, for a time effective to produce the therapeutic agent in the one or more hepatic cells of the mammal. In certain embodiments, the therapeutic agent is stably expressed in a hepatic cell.

In some embodiments, the therapeutic agent is stably expressed in an erythroid cell or a hematopoietic stem cell (HSC). In particular embodiments, the therapeutic agent is a clotting factor (e.g., Factor VIII and Factor IX) that is stably expressed in a hepatic cell. In other embodiments, the therapeutic agent is a globin (e.g., human β-globin and human γ-globin) that is stably expressed in an HSC.

To achieve appropriate expression levels of the protein or polypeptide of interest, any of a number of promoters suitable for use in the selected host cell may be employed. The promoter may be, for example, a constitutive promoter, tissue-specific promoter, inducible promoter, or a synthetic promoter. In various embodiments, the disclosed promoter is a tissue-specific promoter. In some embodiments, the promoter is not a tissue-specific promoter.

Inducible promoters and/or regulatory elements may also be contemplated for achieving appropriate expression levels of the protein or polypeptide of interest. In some embodiments, the promoter of the disclosed rAAV vector and virions is selected from a β-globin promoter, a human parvovirus B19 promoter, a transthyretin (TTR) promoter, or an al anti-trypsin promoter. In some embodiments, the disclosed promoter is a promoter that mediates expression in erythroid cells and/or erythroid lineage cells (e.g., erythroid-specific or blood tissue-specific promoters). In particular embodiments, the disclosed promoter is a β-globin promoter or a human parvovirus B19 promoter. In other embodiments, the disclosed promoter is a promoter that mediates expression in hepatic cells (e.g., hepatic tissue- or hepatic cell-specific promoters). In particular embodiments, the promoter is a transthyretin (TTR) promoter or an al anti-trypsin (AAT) promoter. In some embodiments, the disclosed promoter is active in muscle cells, such as primary human skeletal muscle cells. In some embodiments, the promoter is a muscle creatine kinase promoter (MCK) or a variant thereof (e.g., tMCK).

Other non-limiting examples of suitable inducible promoters include the chicken β-actin (CBA) promoter and promoters from genes such as cytochrome P450 genes, heat shock protein genes, metallothionein genes, and hormone-inducible genes, such as the estrogen gene promoter. In certain embodiments, the AAV vectors described herein comprise a CBA promoter.

In some embodiments, the mammal is a human. In some embodiments, the human is a neonate, a newborn, an infant, or a juvenile. In the practice of the present disclosure, it is contemplated that suitable patients will include, for example, humans that have, are suspected of having, are at risk for developing, or have been diagnosed with, one or more hepatic disorders, diseases, or dystrophies, including, without limitation, hepatic disorders, diseases, and dystrophies that are genetically linked, or inheritable. In some embodiments, suitable patients are at risk for developing, or have been diagnosed with, Duchenne muscular dystrophy.

In some embodiments, the production of the therapeutic agent in the cells targeted for administration of the therapeutic construct preserves or restores function in one or more hepatic cells. In some embodiments, the production of the therapeutic agent in the cells targeted for administration of the therapeutic construct preserves or restores function in one or more hematopoetic cells.

In treating some diseases, it may be preferable to administer the rAAV vector construct a single time, while in the management or treatment of other diseases or conditions, it may be desirable to provide two or more administrations of the vector constructs to the patient in one or more administration periods. In such circumstances, the AAV vector-based therapeutics may be provided successively in one or more daily, weekly, monthly, or less-frequent periods, as may be necessary to achieve treatment, or amelioration of one or more symptoms of the disease or disorder being treated. In some embodiments, the vector is a self-complementary rAAV (scAAV) vector, while in other embodiments, the vector may be provided to the one or both eyes by one or more administrations of an infectious adeno-associated viral particle, an rAAV virion, or a plurality of infectious rAAV particles in an amount and for a time sufficient to treat or ameliorate one or more symptoms of the disease or condition being treated.

In particular embodiments, the disclosure provides improved rAAV particles that have been derived from a number of different serotypes, including, for example, those selected from the group consisting of AAV1, AAV2, AAV3, AAV4, AAV5, AAV6, AAV7, AAV8, AAV9, AAV10, and AAVrh.74. In some embodiments, the AAV particle does not comprise a capsid of serotype 2. In some embodiments, the AAV particle does not comprise a capsid of serotype 5, 8 or 9. In some embodiments, the AAV particle does not comprise a capsid of serotype 1, 4, 7, or 10.

As discussed above, the co-administration methods disclosed herein are suitable for delivery of, and enhance the transduction capacity of, any capsid having cationic patches of amino acid residues on its surface. These capsids include AAV3, AAV6, and capsids derived from AAV3 and AAV6. In addition, such capsids include AAV5, AAV8, AAVrh.74, AAV9, AAV3b, AAVLK03, AAV7BP2, AAV1(E531K), AAV6(D532N), and AAV6-3pmut.

A triple AAV6 mutant (Y445F+Y731F+F129L) was shown to exhibit 101-fold and 49-fold greater transduction efficiencies than wild-type AAV6 in mouse muscle and lung cells, respectively, further exhibiting a 10-fold increase in resistance to neutralization by pooled human immunoglobulins. See van Lieshout et al., A Novel Triple-Mutant AAV6 Capsid Induces Rapid and Potent Transgene Expression in the Muscle and Respiratory Tract of Mice, Mol. Ther. Methods Clin. Dev. 2018; 9: 323-329, herein incorporated by reference. In addition, a quadruple AAV6 mutant (Y705F+Y731F+T492V+K531R) exhibited capability to evade pre-existing antibodies in human K562 cells. For more information on the quadruple AAV6 mutant, see Ling et al., High-Efficiency Transduction of Primary Human Hematopoietic Stem/Progenitor Cells by AAV6 Vectors: Strategies for Overcoming Donor-Variation and Implications in Genome Editing, Sci. Reports, 6: 35495 (2016) and U.S. Patent Publication No. 2014/0341852, published Nov. 20, 2014, each of which is herein incorporated by reference.

Accordingly, in some embodiments, the rAAV particle is a AAV6 particle. In some embodiments, the AAV6 particle comprises a modified capsid protein comprising a non-tyrosine residue at a position that corresponds to a surface-exposed tyrosine residue in a wild-type AAV6 capsid protein, a non-threonine residue at a position that corresponds to a surface-exposed threonine residue in the wild-type AAV6 capsid protein, a non-lysine residue at a position that corresponds to a surface-exposed lysine residue in the wild-type AAV6 capsid protein, a non-serine residue at a position that corresponds to a surface-exposed serine residue in the wild-type AAV6 capsid protein, or a combination thereof. In some embodiments, the modified capsid protein comprises a non-tyrosine residue and/or a non-threonine residue at one or more of or each of Y705, Y731, and T492 of a wild-type AAV6 capsid protein. In some embodiments, the modified capsid protein comprises a non-tyrosine residue and/or a non-threonine residue and/or a non-serine residue at one or more of or each of Y705, Y731, T492 and S663 of a wild-type AAV6 capsid protein. In some embodiments, the modified capsid protein comprises Y705F, Y731F, T492V and/or K531R substitutions. In some embodiments, the non-tyrosine residue is phenylalanine and the non-threonine residue is valine. In some embodiments, the modified capsid protein comprises Y445F, Y731F and/or F129L substitutions. In some embodiments, the modified capsid protein comprises AAV6QM or AAV6TM.

In other embodiments, the modified capsid protein comprises a non-tyrosine (e.g., a phenylalanine) residue at one or more of or each of Y705 and Y731 of a wild-type AAV3 capsid protein. In some embodiments, the modified capsid protein comprises a non-serine residue (e.g., valine) and/or a non-threonine residue (e.g., valine) at one or more of or each of S663 and T492 of a wild-type AAV3 capsid protein. In some embodiments, the modified capsid protein comprises a non-serine residue (e.g., valine), a non-threonine residue (e.g., valine), and/or a non-lysine residue (e.g., arginine) at one or more of or each of S663, T492V and K533 of a wild-type AAV3 capsid protein. In some embodiments, the modified capsid protein comprises a non-tyrosine (e.g., a phenylalanine) residue, non-serine residue (e.g., valine), a non-threonine residue (e.g., valine), and/or a non-lysine residue (e.g., arginine) at one or more of or each of Y705, Y731, S663, T492V and K533 of a wild-type AAV3 capsid protein.

The AAVSHh10 and AAV6(D532N) capsids, both derivatives of AAV6, are described in Klimczak et al., (2009) A Novel Adeno-Associated Viral Variant for Efficient and Selective Intravitreal Transduction of Rat Muller Cells. PLoS ONE 4(10): e746, herein incorporated by reference. The AAV6-3pmut (also known as AAV6(TM6) and AAV6(Y705+Y731F+T492V)) capsid is described in Rosario et al., Microglia-specific targeting by novel capsid-modified AAV6 vectors, Mol Ther Methods Clin Dev. 2016; 13; 3:16026 and International Patent Publication No. 2016/126857, each of which are herein incorporated by reference.

Additional capsids suitable for use with the disclosed methods and compositions include capsids comprising non-native amino acid substitutions at amino acid residues of a wild-type AAV6 capsid, wherein the non-native amino acid substitutions comprise one or more of Y445F, Y705F, Y731F, T492V and S663V.

Additional AAV vectors suitable for use with the disclosed methods and compositions include ITR-modified AAV6 and AAV3 vectors as described in International Publication No. WO 2020/210714, published Oct. 15, 2020, which is herein incorporated by reference.

In other embodiments, the capsid comprises a non-native amino acid substitution at amino acid residue 533 of a wild-type AAV8 capsid, wherein the non-native amino acid substitution is E533K, Y733F, or a combination thereof. The AAV8(Y733F) capsid is described in Doroudchi et al., Amer. Soc. of Gene & Cell Ther. 19(7): 1220-29 (2011). In certain embodiments of the disclosed methods, the capsid comprises AAV7BP2, a variant of AAV8.

In other embodiments, the rAAV particles may comprise a capsid selected from AAV6(3pMut). In other embodiments, the capsid comprises non-native amino acid substitutions of a wild-type AAV6 capsid, comprising one or more of:

(a) Y445F;

(b) Y705F+Y731F;

(c) T492V;

(d) Y705F+Y731F+T492V;

(e) S663V; or

(f) S663V+T492V.

The below tables summarize exemplary capsid variants contemplated for any of the disclosed rAAV viral vectors and particles described herein. In some embodiments, the rAAV particles for use with the methods of administration provided herein comprise a capsid variant selected from any one of Tables 2-12. In some embodiments, the rAAV particles for use with the methods of administration provided herein comprise a capsid variant selected from any one of Tables 2-4 and 11. In some embodiments, the rAAV particles comprise a capsid variant selected from Table 2. In some embodiments, the rAAV particles comprise a capsid variant selected from Table 3. In some embodiments, the rAAV particles comprise a capsid variant selected from Table 4. In some embodiments, the rAAV particles comprise a capsid variant selected from Table 11.

TABLE 2
Mutations of surface-exposed tyrosine (Y), serine (S), threonine
(T), and lysine (K) residues on the AAV3 capsid.

| Single | | |
|---|---|---|
| Mutations | Double Mutations | Multiple Mutations |
| Y252F | Y701 + 705F | Y701 + 705 + 731F |
| Y272F | Y701 + 731F | Y705 + 731F + S663V |
| Y444F | Y705 + 731F | Y705 + 731F + T492V |
| F501Y | S663V + T492V | Y705 + 731F + K533R |
| Y701F | | S663V5 + T492V + K533R |
| Y705F | | Y705 + 731F + S663V + T492V |
| Y731F | | Y705 + 731F + S663V + T492V + |
| | | K533R |
| S459V | | |
| S663V | | |
| T251V | | |
| T492V | | |
| K528E | | |
| K528R | | |
| K533E | | |
| K533R | | |
| K545E | | |
| K545R | | |

TABLE 3
Mutations of surface-exposed tyrosine (Y), serine (S), threonine
(T), and lysine (K) residues on the AAV6 capsid.

| Single | | |
|---|---|---|
| Mutations | Double Mutations | Multiple Mutations |
| Y445F | Y705 + 731F | Y445 + 705 + 731F |
| Y705F | Y731F + T492V | Y705 + 731F + S663V |
| Y731F | T492V + S663V | Y705 + 731F + S551V |
| S455V | T492V + S551V | Y705 + 731F + T492V |
| S472V | T665V + S551V | Y705 + 731F + K531E |
| S499V | S663V + S551V | Y705 + 731F + K666E |
| S551V | T492V + K531E | T492V + S663V + K531E |
| S663V | S663V + K531E | Y705 + 731F + T492V + S663V |
| S669V | T492V + K666E | Y705 + 731F + T492V + K531E |
| T251V | | |
| T492V | | |
| T665V | | |
| T722V | | |
| K531E | | |
| K567E | | |
| K666E | | |
| K707E | | |

TABLE 4
Mutations of surface-exposed tyrosine (Y), threonine
(T), and lysine (K) residues on the AAV8 capsid.

| Single | | |
|---|---|---|
| Mutations | Double Mutations | Multiple Mutations |
| Y253F | Y447 + 733F | Y275 + Y447 + Y733F |
| Y275F | Y733F + T494V | Y253 + Y275 + Y447 + Y703 + |
| | | Y707 + Y733F |
| Y447F | K547 + 569E | Y447 + 733F + T494V |
| Y703F | | |
| Y707F | | |
| Y733F | | |
| T494V | | |
| K137E | | |
| K137R | | |
| K530E | | |
| K547E | | |
| K569E | | |
| K137R | | |

TABLE 5
Mutations of surface-exposed tyrosine (Y), serine (S), threonine
(T), and lysine (K) residues on the AAV1 capsid.

| Single | | |
|---|---|---|
| Mutations | Double Mutations | Multiple Mutations |
| Y252F | Y705 + 731F | Y445 + 705 + 731F |
| Y273F | Y445 + 705F | Y705 + 731F + T492V |
| Y445F | T492V + S663V | Y705 + 731F + S663V |
| Y701F | | Y252 + 445 + 705 + 731F |
| Y705F | | Y273 + 445 + 705 + 731F |
| Y731F | | Y445 + 701 + 705 + 731F |
| S663V | | |
| T251V | | |
| T492V | | |

TABLE 6
Mutations of surface-exposed tyrosine (Y), threonine
(T), and lysine (K) residues on the AAV2 capsid.

| Single | | |
|---|---|---|
| Mutations | Double Mutations | Multiple Mutations |
| Y252F | Y252 + 730F | Y444 + 500 + 730F |
| Y272F | Y272 + 730F | S458 + 492 + 662V |
| Y444F | Y444 + 730F | T455 + 550 + 491V |
| Y500F | Y500 + 730F | T550 + 659 + 491V |
| Y700F | Y700 + 730F | Y730F + S662V + T491V |
| Y704F | Y704 + 730F | K490R + T491V + S492V |
| Y730F | Y444 + 550F | T455 + 550 + 659 + 491V |
| S261V | S458 + 492V | Y272 + 444 + 500 + 730F |
| S264V | S458 + 662V | Y444 + 500 + 730F + S662V |
| S267V | S492 + 662V | Y444 + 500 + 730F + T491V |
| S276V | T455 + 491V | Y444 + 500 + 730F + S458V |
| S384V | T550 + 491V | Y444 + 500 + 730F + K556E |
| S458V | T659 + 491V | Y272 + 444 + 500 + 700 + 730F |
| S468V | T671 + 491V | Y272 + 444 + 500 + 704 + 730F |
| S492V | Y730F + T491V | Y272 + 444 + 500 + 730F + T491V |
| S498V | S662V + T491V | Y444 + 500 + 730F + S662V + |
| | | T491V |
| S578V | Y730F + S662V | Y444 + 500 + 730F + T550 + 491V |
| S658V | K544 + 556E | Y444 + 500 + 730F + T659 + 491V |
| S662V | | Y252 + 272 + 444 + 500 + 704 + |
| | | 730F |
| S662A | | Y444 + 500 + 730F + T455 + 550 + |
| | | 491V |
| S662D | | Y444 + 500 + 730F + S662V + |
| | | T491V + K556E |
| S662F | | Y252 + 272 + 444 + 500 + 700 + |
| | | 704 + 730F |
| S662H | | |
| S662N | | |
| S662L | | |
| S662I | | |
| S668V | | |
| S707V | | |
| S721V | | |
| T251V | | |
| T329V | | |
| T330V | | |
| T454V | | |
| T455V | | |
| T503V | | |
| T550V | | |
| T592V | | |
| T581V | | |
| T597V | | |
| T491V | | |
| T671V | | |
| T659V | | |
| T660V | | |
| T701V | | |
| T713V | | |
| T716V | | |
| K258R | | |
| K321R | | |
| K490R | | |
| K507R | | |
| K527R | | |
| K532R | | |
| K544R | | |
| K459R | | |
| K556R | | |
| K655R | | |
| K649R | | |
| K706R | | |
| K544E | | |
| K556E | | 100 |

TABLE 7
Mutations of surface-exposed tyrosine (Y), threonine
(T), and lysine (K) residues on the AAV4 capsid.

| | | |
|---|---|---|
| Single | | |
| Mutations | Double Mutations | Multiple Mutations |

Y703F
Y729F

TABLE 8
Mutations of surface-exposed tyrosine (Y), serine (S), threonine
(T), and lysine (K) residues on the AAV5 capsid.

| Single | | |
|---|---|---|
| Mutations | Double Mutations | Multiple Mutations |
| Y263F | Y263 + 719F | Y263 + 693 + 719F |
| Y436F | Y436 + 719F | Y436 + 693 + 719F |
| Y693F | Y693 + 719F | Y263 + 436 + 719F |
| Y719F | Y263 + Y436F | |
| S479V | | |
| S651V | | |
| K136E | | |
| K136R | | |
| K251E | | |
| K251R | | |

TABLE 9
Mutations of surface-exposed tyrosine (Y), threonine
(T), and lysine (K) residues on the AAV7 capsid.

| | | |
|---|---|---|
| Single | | |
| Mutations | Double Mutations | Multiple Mutations |

Y253F
Y273F
Y446F
Y702F
Y733F

TABLE 10
Mutations of surface-exposed tyrosine (Y), serine (S), threonine
(T), and lysine (K) residues on the AAV9 capsid.

| Single | | |
|---|---|---|
| Mutations | Double Mutations | Multiple Mutations |
| Y705F | Y705 + 731F | Y705 + 731F + T492V |
| Y731F | | |
| T492V | | |

TABLE 11
Mutations of surface-exposed tyrosine (Y), threonine
(T), and lysine (K) residues on the AAVrh.74 capsid.

| Single | | |
|---|---|---|
| Mutations | Double Mutations | Multiple Mutations |
| Y447F | Y447 + 733F | Y447 + 733F + N665R |
| Y733F | | Y447 + 733F + T494V |
| N665R | | Y447 + 733F + K5475R |
| T494V | | Y447 + 733F + N665R + |
| | | T494V + K547R |
| K547R | | |

TABLE 12
Mutations of surface-exposed tyrosine (Y), serine (S), threonine
(T), and lysine (K) residues on the AAV10 capsid.

| | | |
|---|---|---|
| Single | | |
| Mutations | Double Mutations | Multiple Mutations |

Y447F
Y733F

The first letter corresponds to the amino acid in the wild-type AAV capsid, the number is the VP3 amino acid position that was mutated, and the last letter is the mutant amino acid.

The rAAV polynucleotide or nucleic acid vectors of the present disclosure may be comprised within a virion having a serotype that is selected from the group consisting of AAV serotype 1, AAV serotype 2, AAV serotype 3, AAV serotype 4, AAV serotype 5, AAV serotype 6, AAV serotype 7, AAV serotype 8, AAV serotype 9, or AAV serotype 10, or any other serotype as known to one of ordinary skill in the viral arts.

In related embodiments, the disclosure further provides populations and pluralities of rAAV polynucleotide or nucleic acid vectors, virions, infectious viral particles, or host cells that comprise a multi-mutated capsid protein and one or more nucleic acid segments that include a promoter operably linked to a selected polynucleotide encoding at least a first diagnostic and/or a first therapeutic molecule.

The disclosure further provides composition and formulations that include one or more of the proteins, nucleic acid segments, viral polynucleotide or nucleic acid vectors, host cells, or viral particles of the present disclosure together with one or more pharmaceutically-acceptable buffers, diluents, or excipients. Such compositions may be included in one or more diagnostic or therapeutic kits, for diagnosing, preventing, treating or ameliorating one or more symptoms of a mammalian disease, injury, disorder, trauma or dysfunction, and in particular, for delivery of a therapeutic agent to hepatic cells or HSCs of a subject (e.g., a human subject). In particular embodiments, the therapeutic agent is a clotting factor (e.g., Factor VIII and Factor IX) that is stably expressed in a hepatic cell. In other embodiments, the therapeutic agent is a globin (e.g., human β-globin and human γ-globin) that is stably expressed in a HSC.

The disclosure further includes a method for providing a mammal in need thereof with a diagnostically- or therapeutically-effective amount of a selected therapeutic agent, the method comprising providing to a cell, tissue or organ of a mammal in need thereof, an amount of one or more of the disclosed rAAV multi-capsid mutant particles or nucleic acid vectors; and for a time effective to provide the mammal with a diagnostically- or a therapeutically-effective amount of the selected therapeutic agent.

The disclosure further provides a method for diagnosing, preventing, treating, or ameliorating at least one or more symptoms of a disease, a disorder, a dysfunction, an injury, an abnormal condition, or trauma in a mammal. In an overall and general sense, the method includes at least the step of administering to a mammal in need thereof one or more of the disclosed rAAV particles or nucleic acid vectors, in an amount and for a time sufficient to diagnose, prevent, treat or ameliorate the one or more symptoms of the disease, disorder, dysfunction, injury, abnormal condition, or trauma in the mammal.

The disclosure also provides a method of transducing a population of mammalian cells, and particular one or more cells in the human liver cells and/or HSCs. In an overall and general sense, the method includes at least the step of introducing into one or more cells of the population, a composition that comprises an effective amount of one or more of the rAAV particles or nucleic acid vectors disclosed herein. In certain embodiments, delivery of the disclosed gene therapy constructs to one or more cells hepatically, permitting the high-efficiency transduction of human liver cells and/or HSCs. In exemplary embodiments, delivery of any of the disclosed rAAV particle compositions are to hepatic blood vessels of the subject.

In a further embodiment, the disclosure also provides isolated nucleic acid segments that encode one or more of the AAV mutant capsid proteins as described herein, and provides recombinant nucleic acid vectors comprising said segments.

Additionally, the present disclosure provides compositions, as well as therapeutic and/or diagnostic kits that include one or more of the disclosed AAV particle or nucleic acid vector compositions, formulated with one or more additional ingredients, or prepared with one or more instructions for their use.

In one aspect, the disclosure provides compositions comprising recombinant adeno-associated viral (AAV) vectors, virions, viral particles, and pharmaceutical formulations thereof, useful in methods for delivering genetic material encoding one or more beneficial or therapeutic product(s) to mammalian cells and tissues. In particular, the compositions and methods of the disclosure provide a significant advancement in the art through their use in the treatment, prevention, and/or amelioration of symptoms of one or more mammalian diseases. It is contemplated that human gene therapy will particularly benefit from the present teachings by providing new and improved viral vector constructs for use in the treatment of a number of diverse diseases, disorders, and dysfunctions. In certain embodiments, any one of the disclosed rAAV vectors and virions comprise a polynucleotide that encodes a clotting factor (e.g., Factor VIII and Factor IX). In other embodiments, any one of the disclosed rAAV vectors and virions comprise a polynucleotide that encodes a globin (e.g., human β-globin and human γ-globin).

In some embodiments, the rAAV particles of the disclosed methods comprises a vector comprising a heterologous nucleic acid sequence that encodes at least a first diagnostic or therapeutic agent operably linked to a hepatocyte-specific promoter capable of expressing the segment in one or more liver cells that have been transformed with the vector. In other embodiments, the rAAV particles of the disclosed methods comprises a vector comprising a heterologous nucleic acid sequence that encodes at least a first diagnostic or therapeutic agent operably linked to a blood tissue-specific promoter capable of expressing the segment in one or more liver cells that have been transformed with the vector.

The surface-exposed amino acid-modified rAAV particles or nucleic acid vectors of the present disclosure may optionally further include one or more enhancer sequences that are each operably linked to the nucleic acid segment that encodes the diagnostic or therapeutic molecule of interest. Exemplary enhancer sequences include, but are not limited to, one or more selected from the group consisting of a CMV enhancer, a synthetic enhancer; or a liver tissue-specific enhance such as an albumin enhancer or hepatitis B enhancer and such like, and any combination thereof.

Exemplary promoters useful in the practice of the disclosure include, without limitation, one or more tissue-specific promoters. In some embodiments, exemplary promoters are mammalian tissue specific. In particular embodiments, the disclosed promoters are mammalian liver tissue-specific promoters such as a transthyretin (TTR) promoter or an al anti-trypsin promoter. In other embodiments, the disclosed promoters are mammalian erthroid tissue-specific promoters such a β-globin promoter or a human parvovirus B19 promoter.

The nucleic acid vector may also further include one or more post-transcriptional regulatory sequences or one or more polyadenylation signals, including, for example, but not limited to, a woodchuck hepatitis virus post-transcription regulatory element (WRPE), a polyadenylation signal sequence, or an intron/exon junctions/splicing signals, or any combination thereof.

In exemplary embodiments, the improved rAAV particles of the disclosure will encode at least one diagnostic or therapeutic protein or polypeptide selected from the group consisting of a clotting factor, a globin molecular marker, photosensitive opsins, including, without limitation, rhodopsin, melanopsin, cone opsins, channel rhodopsins, bacterial or archea-associated opsins, an adrenergic agonist, an anti-apoptosis factor, an apoptosis inhibitor, a cytokine receptor, a cytokine, a cytotoxin, an erythropoietic agent, a glutamic acid decarboxylase, a glycoprotein, a growth factor, a growth factor receptor, a hormone, a hormone receptor, an interferon, an interleukin, an interleukin receptor, a kinase, a kinase inhibitor, a nerve growth factor, a netrin, a neuroactive peptide, a neuroactive peptide receptor, a neurogenic factor, a neurogenic factor receptor, a neuropilin, a neurotrophic factor, a neurotrophin, a neurotrophin receptor, an N-methyl-D-aspartate antagonist, a plexin, a protease, a protease inhibitor, a protein decarboxylase, a protein kinase, a protein kinsase inhibitor, a proteolytic protein, a proteolytic protein inhibitor, a semaphorin, a semaphorin receptor, a serotonin transport protein, a serotonin uptake inhibitor, a serotonin receptor, a serpin, a serpin receptor, a tumor suppressor, and any combination thereof. In particular embodiments, the rAAV particles encode a clotting factor or a globin.

In another embodiment, the disclosure concerns rAAV nucleic acid vectors that include at least a first nucleic acid segment that encodes one or more diagnostic or therapeutic agents that alter, inhibit, reduce, prevent, eliminate, or impair the activity of one or more endogenous biological processes in a mammalian cell suitably transformed with the vector of interest. In certain embodiments, such diagnostic or therapeutic agents may include a molecule that selectively inhibits or reduces the effects of one or more metabolic processes, dysfunctions, disorders, or diseases. In certain embodiments, the defect may be caused by injury or trauma to the mammal for which treatment is desired. In other embodiments, the defect may be caused the over-expression of an endogenous biological compound, while in other embodiments still; the defect may be caused by the under-expression or even lack of one or more endogenous biological compounds.

The rAAV nucleic acid vectors and expression systems of the present disclosure may also further include a second nucleic acid segment that comprises, consists essentially of, or consists of, one or more enhancers, one or more regulatory elements, one or more transcriptional elements, or any combination thereof, that alter, improve, regulate, and/or affect the transcription of the nucleotide sequence of interest expressed by the rAAV particles.

For example, the rAAV nucleic acid vectors of the present disclosure may further include a second nucleic acid segment that comprises, consists essentially of, or consists of, a CMV enhancer, a synthetic enhancer, a cell-specific enhancer, a tissue-specific enhancer, or any combination thereof. The second nucleic acid segment may also further comprise, consist essentially of, or consist of, one or more intron sequences, one or more post-transcriptional regulatory elements, or one or more enhancers from rhodopsin, melanopsin, cone opsins, channel rhodopsins, bacterial or archea-associated opsins, an adrenergic agonist, an anti-apoptosis factor, an apoptosis inhibitor, a cytokine receptor, a cytokine, a cytotoxin, an erythropoietic agent, a glutamic acid decarboxylase, a glycoprotein, a growth factor, a growth factor receptor, a hormone, a hormone receptor, an interferon, an interleukin, an interleukin receptor, a kinase, a kinase inhibitor, a nerve growth factor, a netrin, a neuroactive peptide, a neuroactive peptide receptor, a neurogenic factor, a neurogenic factor receptor, a neuropilin, a neurotrophic factor, a neurotrophin, a neurotrophin receptor, an N-methyl-D-aspartate antagonist, a plexin, a protease, a protease inhibitor, a protein decarboxylase, a protein kinase, a protein kinase inhibitor, a proteolytic protein, a proteolytic protein inhibitor, a semaphorin, a semaphorin receptor, a serotonin transport protein, a serotonin uptake inhibitor, a serotonin receptor, a serpin, a serpin receptor, or a tumor suppressor.

The particles of the present disclosure may also optionally further include a polynucleotide that comprises, consists essentially of, or consists of, one or more polylinkers, restriction sites, and/or multiple cloning region(s) to facilitate insertion (cloning) of one or more selected genetic elements, genes of interest, and/or one or more therapeutic or diagnostic molecules into the rAAV particle at a selected site within the vector.

In further aspects of the present disclosure, the exogenous polynucleotide(s) that may be delivered into suitable host cells by the rAAV particles comprising nucleic acid vectors disclosed herein are of mammalian origin, with polynucleotides encoding one or more polypeptides or peptides of, e.g., human, non-human primate, porcine, bovine, ovine, feline, canine, equine, epine, caprine, or lupine origin.

The exogenous polynucleotide(s) that may be delivered into host cells by the disclosed particles or viral vectors may, in certain embodiments, encode one or more proteins, one or more polypeptides, one or more peptides, one or more enzymes, or one or more antibodies (or antigen-binding fragments thereof), or alternatively, may express one or more siRNAs, ribozymes, antisense oligonucleotides, PNA molecules, or any combination thereof. When combinational gene therapies are desired, two or more different molecules may be produced from a single rAAV expression system, or alternatively, a selected host cell may be transfected with two or more unique rAAV expression systems, each of which may comprise one or more distinct polynucleotides that encode a therapeutic agent. In particular embodiments, the therapeutic agent is selected from a globin and a clotting factor.

In other embodiments, the disclosure also provides rAAV nucleic acid vectors that are comprised within an infectious adeno-associated viral particle or a virion, as well as pluralities of such virions or infectious particles. Such vectors and virions may be comprised within one or more diluents, buffers, physiological solutions or pharmaceutical vehicles, or formulated for administration to a mammal in one or more diagnostic, therapeutic, and/or prophylactic regimens. The vectors, virus particles, virions, and pluralities thereof of the present disclosure may also be provided in excipient formulations that are acceptable for veterinary administration to selected livestock, exotics, domesticated animals, and companion animals (including pets and such like), as well as to non-human primates, zoological or otherwise captive specimens.

Other aspects of the disclosure concern recombinant adeno-associated virus virion particles (e.g., improved transduction efficiency particles), compositions, and host cells that comprise, consist essentially of, or consist of, one or more of the rAAV particles disclosed herein, such as for example pharmaceutical formulations of the vectors intended for administration to a mammal (e.g., a human).

Kits comprising one or more of the disclosed rAAV particles or nucleic acid vectors (as well as one or more virions, viral particles, transformed host cells or pharmaceutical compositions comprising such vectors); and instructions for using such kits in one or more therapeutic, diagnostic, and/or prophylactic clinical embodiments are also provided by the present disclosure. Such kits may further comprise one or more reagents, restriction enzymes, peptides, therapeutics, pharmaceutical compounds, or means for delivery of the composition(s) to host cells, or to an animal (e.g., syringes, injectables, and the like). Exemplary kits include those for treating, preventing, or ameliorating the symptoms of a disease, deficiency, dysfunction, and/or injury, or may include components for the large-scale production of the viral vectors themselves, such as for commercial sale, or for use by others, including e.g., virologists, medical professionals, and the like.

Another aspect of the present disclosure concerns methods of use of the disclosed rAAV particles or vectors, virions, expression systems, compositions, and host cells described herein in the preparation of medicaments for diagnosing, preventing, treating or ameliorating at least one or more symptoms of a disease, a dysfunction, a disorder, an abnormal condition, a deficiency, injury, or trauma in an animal, and in particular, in the eye of a vertebrate mammal. Such methods generally involve direct administration to the liver or blood of a mammal in need thereof, one or more of the disclosed vectors, virions, viral particles, host cells, compositions, or pluralities thereof, in an amount and for a time sufficient to diagnose, prevent, treat, or lessen one or more symptoms of such a disease, dysfunction, disorder, abnormal condition, deficiency, injury, or trauma in one or both eyes of the affected animal. The methods may also encompass prophylactic treatment of animals suspected of having such conditions, or administration of such compositions to those animals at risk for developing such conditions either following diagnosis, or prior to the onset of symptoms.

Compositions comprising one or more of the disclosed rAAV particles, expression systems, infectious AAV particles, host cells also form part of the present disclosure, and particularly those compositions that further comprise at least a first pharmaceutically-acceptable excipient for use in the manufacture of medicaments and methods involving therapeutic administration of such rAAV particles or nucleic acid vectors. In some embodiments, pharmaceutical formulations are suitable for administration to a human or other mammal.

Another aspect of the present disclosure concerns methods of use of the particles, vectors, virions, expression systems, compositions, and host cells described herein in a method for treating or ameliorating the symptoms or in the preparation of medicaments for treating or ameliorating the symptoms of various deficiencies in mammalian liver tissues, such as human liver tissues. Exemplary diseases and disorders of the liver for treatment or amelioration of symptoms include liver cancers such as hepatocellular carcinoma (HCC), cholangiocarcinoma, angiosarcoma, and hepatoblastoma.

Another aspect of the present disclosure concerns methods of use of the particles, vectors, virions, expression systems, compositions, and host cells described herein in a method for treating or ameliorating the symptoms or in the preparation of medicaments for treating or ameliorating the symptoms of a hemoglobinopathy. A hemoglobinopathy is a disease or disorder characterized by one or more mutation(s) in the genome that results in abnormal structure of one or more of the globin chains of the hemoglobin molecule. Exemplary hemoglobinopathies include hemolytic anemia, sickle cell disease, and thalassemia. Sickle cell disease is characterized by the presence of abnormal, sickle-chalped hemoglobins, which can result in severe infections, severe pain, stroke, and an increased risk of death. Subjects having sickle cell disease can be identified, e.g., using one or more of a complete blood count, a blood film, hemoglobin electrophoresis, and genetic testing. Thalassemias are a group of autosomal recessive diseases characterized by a reduction in the amount of hemoglobin produced. Symptoms include iron overload, infection, bone deformities, enlarged spleen, and cardiac disease. The subgroups of thalassemias include alpha-thalassemia, beta-thalassemia, and delta thalassemia. Additional hemoglobinopathies such as hemophilia A and hemophilia B may be treated using any of the particles, vectors, virions, expression systems, compositions, and host cells described herein.

Such methods may involve administration to a subject in need thereof, one or more of the disclosed particles vectors, virions, host cells, or compositions, into the hepatic blood vessels of the subject in an amount and for a time sufficient to treat or ameliorate the symptoms of such a deficiency in the affected mammal. The methods may also encompass prophylactic treatment of animals suspected of having such conditions, or administration of such compositions to those animals at risk for developing such conditions either following diagnosis, or prior to the onset of symptoms.

In various embodiments, the compositions and methods of use described herein do not comprise use of any adenoviral vectors or adenoviral particles. In various embodiments, the compositions and methods of use described herein do not comprise use of any naked DNA plasmids.

### Storage and Manufacturing Buffers

In other aspects, provided herein are buffers for storage and manufacturing of rAAV particles.

Foremost, provided herein are buffers for storage of a mixture of rAAV vectors or capsids and PVA. In some embodiments, the disclosed buffers comprise PVA in a concentration of 0.01%, 0.05%, 0.1%, 0.2%, 0.3%, 0.4%, 0.5%, 0.6%, 0.75%, 0.9%, 1.0%, 1.25%, 1.5%, 1.75%, 2.0%, 2.25%, 2.5%, 2.75%, 3.0%, 3.25%, 3.5%, 3.75%, 4.0%, 5.0%, or 6.0% weight by volume (w/v) and BSS. In some embodiment, the disclosed buffers comprise PVA in a concentration of 0.01%, 0.05%, 0.1%, 0.2%, 0.3%, 0.4%, 0.5%, 0.6%, 0.75%, 0.9%, 1.0%, 1.25%, 1.5%, 1.75%, 2.0%, 2.25%, 2.5%, 2.75%, 3.0%, 3.25%, 3.5%, 3.75%, 4.0%, 5.0%, or 6.0% w/v and BSS, and one or more of the following excipients: artificial cerebrospinal fluid (CSF), PBS, Ringer's lactate solution, TMN200 solution, polysorbate 20, and poloxamer 188 (known commercially as Pluronic F-68®), and/or additional excipients.

In particular embodiments, the buffer comprises PVA in a concentration of about 0.4% w/v, and one or more of BSS, artificial CSF, and PBS. In additional embodiments, the buffer comprises PVA in a concentration of about 0.4% w/v, and one or more of BSS, artificial CSF, PBS, Ringer's lactate solution; and optionally TMN200 solution; and further optionally polysorbate 20 (Tween 20), and poloxamer 188. In particular embodiments, the disclosed buffers comprise Tween 20 in a concentration of about 0.014% and poloxamer 100 in a concentration of about 0.01%. In some embodiments, the buffer comprises PVA in a concentration other than about 0.4%.

In certain embodiments, the disclosed buffers consist essentially of PVA in a concentration of about PVA in a concentration of 0.01%, 0.05%, 0.1%, 0.2%, 0.3%, 0.4%, 0.5%, 0.6%, 0.75%, 0.9%, 1.0%, 1.25%, 1.5%, 1.75%, 2.0%, 2.25%, 2.5%, 2.75%, 3.0%, 3.25%, 3.5%, 3.75%, 4.0%, 5.0%, or 6.0% w/v, BSS, artificial CSF, PBS, Ringer's lactate solution and TMN200 solution.

Further provided herein are elution buffers for manufacturing rAAV particles that comprise PVA. These buffers may be used to elute AAV capsids from an affinity column immediately prior to packaging and formulation fo a final product. These disclosed buffers may prevent aggregation of AAV capsids having surface-exposed cationic patches from aggregating during manufacturing. These buffers may be of pH lower than 7, optionally lower than 5.

In some embodiments, the disclosed buffers comprise PVA in a concentration of 0.01%, 0.05%, 0.1%, 0.2%, 0.3%, 0.4%, 0.5%, 0.6%, 0.75%, 0.9%, 1.0%, 1.25%, 1.5%, 1.75%, 2.0%, 2.25%, 2.5%, 2.75%, 3.0%, 3.25%, 3.5%, 3.75%, 4.0%, 5.0%, or 7.5% w/v.

In some embodiments, the disclosed buffers comprise PVA that is hydrolyzed to about 80%, 82%, 85%, 87%, 89%, 91%, 93%, 95%, 97%, or 99%. In certain embodiments, the buffers comprise 87% hydrolyzed PVA (PVA87). In certain embodiments, the buffers comprise 99% hydrolyzed PVA (PVA99). In some embodiments, the buffers comprise PVA wherein the PVA is not 87% hydrolyzed.

Further disclosed are methods of manufacturing an rAAV particle comprising purifying AAV capsids wherein capsids are eluted from an affinity column containing a resin selected from AAV-X or AVB affinity resins, or another resin known in the art. See Terova et al. Cell & Gene Therapy Insights, ISSN: 2059-7800 (2018), herein incorporated by reference.

rAAV Particles and Nucleic Acid Vectors

Recombinant adeno-associated virus (rAAV) vectors have been used successfully for in vivo gene transfer in numerous pre-clinical animal models of human disease, and PVAve been used successfully for long-term expression of a wide variety of therapeutic genes (Daya and Berns, 2008; Niemeyer et al., 2009; Owen et al., 2002; Keen-Rhinehart et al., 2005; Scallan et al., 2003; Song et al., 2004). AAV vectors have also generated long-term clinical benefit in humans when targeted to immune-privileged sites, i.e., ocular delivery for Leber's congenital amaurosis (Bainbridge et al., 2008; Maguire et al., 2008; Cideciyan et al., 2008). A major advantage of this vector is its comparatively low immune profile, eliciting only limited inflammatory responses and, in some cases, even directing immune tolerance to transgene products (LoDuca et al., 2009). Nonetheless, the therapeutic efficiency, when targeted to non-immune privileged organs, has been limited in humans due to antibody and CD8⁺ T cell responses against the viral capsid, while in animal models, adaptive responses to the transgene product have also been reported (Manno et al., 2006; Mingozzi et al., 2007; Muruve et al., 2008; Vandenberghe and Wilson, 2007; Mingozzi and High, 2007). These results suggested that immune responses remain a concern for AAV vector-mediated gene transfer.

Aspects of the disclosure relate to recombinant adeno-associated virus (rAAV) particles or preparations of such particles for delivery of one or more nucleic acid vectors comprising a sequence encoding a Rep protein, and/or a protein or polypeptide of interest, into various tissues, organs, and/or cells. In some embodiments, the rAAV particle is delivered to a host cell in the presence of a Rep protein as described herein.

The wild-type AAV genome is a single-stranded deoxyribonucleic acid (ssDNA), either positive- or negative-sensed. The genome comprises two inverted terminal repeats (ITRs), one at each end of the DNA strand, two open reading frames (ORFs): rep and cap between the ITRs, and an insert nucleic acid positioned between the ITRs and optionally comprising a transgene. The rep ORF comprises four overlapping genes encoding Rep proteins required for the AAV life cycle. The cap ORF comprises overlapping genes encoding capsid proteins: VP1, VP2 and VP3, which interact together to form the viral capsid. VP1, VP2 and VP3 are translated from one mRNA transcript, which can be spliced in two different manners: either a longer or shorter intron can be excised resulting in the formation of two isoforms of mRNAs: a ˜2.3 kb- and a ˜2.6 kb-long mRNA isoform. The capsid forms a supramolecular assembly of approximately 60 individual capsid protein subunits into a non-enveloped, T-1 icosahedral lattice capable of protecting the AAV genome. The mature capsid is composed of VP1, VP2, and VP3 (molecular masses of approximately 87, 73, and 62 kDa respectively) in a ratio of about 1:1:10.

Recombinant AAV (rAAV) particles may comprise a nucleic acid vector, which may comprise at a minimum: (a) one or more transgenes comprising a sequence encoding a protein or polypeptide of interest or an RNA of interest (e.g., a siRNA or microRNA), or one or more nucleic acid regions comprising a sequence encoding a Rep protein; and (b) one or more regions comprising inverted terminal repeat (ITR) sequences (e.g., engineered ITR sequences) flanking the one or more nucleic acid regions (e.g., transgenes). In some embodiments, the nucleic acid vector is between 4 kb and 5 kb in size (e.g., 4.2 to 4.7 kb in size). In some embodiments, the nucleic acid vector further comprises a region encoding a Rep protein as described herein. Any nucleic acid vector described herein may be encapsidated by a viral capsid, such as an AAV6 capsid or another serotype (e.g., a serotype that is of the same serotype as the ITR sequences), which may comprises a modified capsid protein as described herein. In some embodiments, the nucleic acid vector is circular. In some embodiments, the nucleic acid vector is single-stranded. In some embodiments, the nucleic acid vector is double-stranded. In some embodiments, a double-stranded nucleic acid vector may be, for example, a self-complimentary vector that contains a region of the nucleic acid vector that is complementary to another region of the nucleic acid vector, initiating the formation of the double-strandedness of the nucleic acid vector.

Accordingly, in some embodiments, an rAAV particle or rAAV preparation containing such particles comprises a viral capsid and a nucleic acid vector as described herein, which is encapsidated by the viral capsid. In some embodiments, the insert nucleic acid of the nucleic acid vector comprises (1) one or more transgenes comprising a sequence encoding a protein or polypeptide of interest, (2) one or more nucleic acid regions comprising a sequence that facilitates expression of the transgene (e.g., a promoter), and (3) one or more nucleic acid regions comprising a sequence that facilitate integration of the transgene (optionally with the one or more nucleic acid regions comprising a sequence that facilitates expression) into the genome of the subject.

By a nucleic acid molecule comprising a nucleotide sequence having at least, for example, 95% “identity” to a query nucleic acid sequence, it is intended that the nucleotide sequence of the subject nucleic acid molecule is identical to the query sequence except that the subject nucleic acid molecule sequence may include up to five nucleotide alterations per each 100 nucleotides of the query sequence. In other words, to obtain a promoter having a nucleotide sequence at least 95% identical to a reference (query) sequence, up to 5% of the nucleotides in the subject sequence may be inserted, deleted, or substituted with another nucleotide. These alterations of the reference sequence may occur at the 5′ or 3′ ends of the reference sequence or anywhere between those positions, interspersed either individually among nucleotides in the reference sequence or in one or more contiguous groups within the reference sequence.

As a practical matter, whether any particular nucleic acid molecule is at least 80%, 85%, 90%, 95%, 96%, 97%, 98%, or 99% identical to, for instance, the nucleotide sequence of an rAAV vector, can be determined conventionally using known computer programs. A preferred method for determining the best overall match between a query sequence (e.g., a sequence of the present disclosure) and a subject sequence, also referred to as a global sequence alignment, can be determined using the FASTDB or blastn computer program based on the algorithm of Brutlag et al. (Comp. App. Biosci. 6:237-245 (1990)). In a sequence alignment the query and subject sequences are either both nucleotide sequences or both amino acid sequences. The result of said global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. Whether a nucleotide is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present disclosure. For subject sequences truncated at the 5′ and/or 3′ ends, relative to the query sequence, the percent identity is corrected by calculating the number of nucleotides of the query sequence that are positioned 5′ to or 3′ to the query sequence, which are not matched/aligned with a corresponding subject nucleotide, as a percent of the total bases of the query sequence.

In some embodiments, the nucleic acid vector comprises one or more transgenes comprising a sequence encoding a protein or polypeptide of interest operably linked to a promoter, wherein the one or more transgenes are flanked on each side with an ITR sequence. In some embodiments, the nucleic acid vector further comprises a region encoding a Rep protein as described herein, either contained within the region flanked by ITRs or outside the region or nucleic acid) operably linked to a promoter, wherein the one or more nucleic acid regions. The ITR sequences can be derived from any AAV serotype (e.g., 1, 2, 3, 4, 5, 6, 7, 8, 9, or 10) or can be derived from more than one serotype. In some embodiments, the ITR sequences are derived from AAV3 or AAV6. In some embodiments, the ITR sequences of the first serotype are derived from AAV3, AAV6 or AAV8/rh.74. In some embodiments, the ITR sequences are the same serotype as the capsid (e.g., AAV3 ITR sequences and AAV3 capsid, etc.).

ITR sequences and plasmids containing ITR sequences are known in the art and commercially available (see, e.g., products and services available from Vector Biolabs, Philadelphia, Pa.; Cellbiolabs, San Diego, Calif.; Agilent Technologies, Santa Clara, Ca; and Addgene, Cambridge, Mass.; and Gene delivery to skeletal muscle results in sustained expression and systemic delivery of a therapeutic protein. Kessler P D, et al. Proc Natl Acad Sci USA. 1996; 93(24):14082-7; and Curtis A. Machida, Methods in Molecular MedicineTM Viral Vectors for Gene Therapy Methods and Protocols. 10.1385/1-59259-304-6:201 Humana Press Inc. 2003: Chapter 10, Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference).

In some embodiments, the nucleic acid vector comprises a pTR-UF-11 plasmid backbone, which is a plasmid that contains AAV2 ITRs. This plasmid is commercially available from the American Type Culture Collection (ATCC MBA-331).

Exemplary rAAV nucleic acid vectors useful according to the disclosure include single-stranded (ss) or self-complementary (sc) AAV nucleic acid vectors, such as single-stranded or self-complementary recombinant viral genomes. In some embodiments, the disclosed vectors are self-complementary AAV vectors. In various embodiments, the vectors are scAAV3, scAAV6, scAAVrh.74, scAAV5, scAAV8, scAAV8, or scAAV9 vectors.

Methods of producing rAAV particles and nucleic acid vectors are also known in the art and commercially available (see, e.g., Zolotukhin et al. Production and purification of serotype 1, 2, and 5 recombinant adeno-associated viral vectors. Methods 28 (2002) 158-167; and U.S. Patent Publication Nos. 2007/0015238 and US 2012/0322861, which are incorporated herein by reference; and plasmids and kits available from ATCC and Cell Biolabs, Inc.). For example, a plasmid containing the nucleic acid vector sequence may be combined with one or more helper plasmids, e.g., that contain a rep gene (e.g., encoding Rep78, Rep68, Rep52 and Rep40) and a cap gene (encoding VP1, VP2, and VP3, including a modified VP3 region as described herein), and transfected into a producer cell line such that the rAAV particle can be packaged and subsequently purified.

In some embodiments, the one or more helper plasmids includes a first helper plasmid comprising a rep gene and a cap gene and a second helper plasmid comprising a Ela gene, a E1b gene, a E4 gene, a E2a gene, and a VA gene. In some embodiments, the rep gene is a rep gene derived from AAV3 and the cap gene is derived from AAV3 and includes modifications to the gene in order to produce a modified capsid protein described herein. In some embodiments, the rep gene is a rep gene derived from AAV6 and the cap gene is derived from AAV6 and includes modifications to the gene in order to produce a modified capsid protein described herein. Helper plasmids, and methods of making such plasmids, are known in the art and commercially available (see, e.g., pDM, pDG, pDP1rs, pDP2rs, pDP3rs, pDP4rs, pDP5rs, pDP6rs, pDG(R484E/R585E), and pDP8.ape plasmids from PlasmidFactory, Bielefeld, Germany; other products and services available from Vector Biolabs, Philadelphia, Pa.; Cellbiolabs, San Diego, Calif.; Agilent Technologies, Santa Clara, Ca; and Addgene, Cambridge, Mass.; pxx6; Grimm et al. (1998), Novel Tools for Production and Purification of Recombinant Adenoassociated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081.; Grimm et al. (2003), Helper Virus-Free, Optically Controllable, and Two-Plasmid-Based Production of Adeno-associated Virus Vectors of Serotypes 1 to 6, Molecular Therapy, Vol. 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R. O. (2008), International efforts for recombinant adeno-associated viral vector reference standards, Molecular Therapy, Vol. 16, 1185-1188).

An exemplary, non-limiting, rAAV particle production method is described next. One or more helper plasmids are produced or obtained, which comprise rep and cap ORFs for the desired AAV serotype and the adenoviral VA, E2A (DBP), and E4 genes under the transcriptional control of their native promoters. The cap ORF may also comprise one or more modifications to produce a modified capsid protein as described herein. HEK293 cells (available from ATCC®) are transfected via CaPO4-mediated transfection, lipids or polymeric molecules such as Polyethylenimine (PEI) with the helper plasmid(s) and a plasmid containing a nucleic acid vector described herein. The HEK293 cells are then incubated for at least 60 hours to allow for rAAV particle production. Alternatively, in another example Sf9-based producer stable cell lines are infected with a single recombinant baculovirus containing the nucleic acid vector. As a further alternative, in another example HEK293 or BHK cell lines are infected with a HSV containing the nucleic acid vector and optionally one or more helper HSVs containing rep and cap ORFs as described herein and the adenoviral VA, E2A (DBP), and E4 genes under the transcriptional control of their native promoters. The HEK293, BHK, or Sf9 cells are then incubated for at least 60 hours to allow for rAAV particle production. The rAAV particles can then be purified using any method known the art or described herein, e.g., by iodixanol step gradient, CsCl gradient, chromatography, or polyethylene glycol (PEG) precipitation.

Kits and Uses for Improved rAAV Delivery

The present disclosure provides one or more of the disclosed mixtures of rAAV particles and polyvinyl alcohol comprised within a kit for diagnosing, preventing, treating or ameliorating one or more symptoms of a mammalian disease, injury, disorder, trauma or dysfunction. Such kits may be useful in diagnosis, prophylaxis, and/or therapy, and particularly useful in the treatment, prevention, and/or amelioration of one or more defects in the mammalian eye as discussed herein.

The disclosure also provides for the use of the buffers and compositions disclosed herein in the manufacture of a medicament for treating, preventing or ameliorating the symptoms of a disease, disorder, dysfunction, injury or trauma, including, but not limited to, the treatment, prevention, and/or prophylaxis of a disease, disorder or dysfunction, and/or the amelioration of one or more symptoms of such a disease, disorder or dysfunction.

Likewise, the disclosure also provides methods for treating or ameliorating the symptoms of such a disease, injury, disorder, or dysfunction in one or both eyes of a mammal, and of a human in particular. Such methods generally involve at least the step of administering to a mammal in need thereof, one or more of the rAAV particles as disclosed herein, in an amount and for a time sufficient to treat or ameliorate the symptoms of such a disease, injury, disorder, or dysfunction in one or both eyes of the mammal.

Pharmaceutical Compositions Comprising rAAV Particles

One important aspect of the present methodology is the fact that the improved rAAV delivery methods described herein permit the delivery of smaller titers of viral particles in order to achieve the same transduction efficiency as that obtained using higher levels of conventional, rAAV methods. To that end, the amount of AAV compositions and time of administration of such compositions will be within the purview of the skilled artisan having benefit of the present teachings. In fact, the inventors contemplate that the administration of therapeutically-effective amounts of the disclosed compositions may be achieved by a single administration, such as for example, a single injection of sufficient numbers of infectious particles to provide therapeutic benefit to the patient undergoing such treatment. Alternatively, in some circumstances, it may be desirable to provide multiple, or successive administrations of the AAV vector compositions, either over a relatively short, or over a relatively prolonged period, as may be determined by the medical practitioner overseeing the administration of such compositions.

For example, the number of infectious particles administered to a mammal may be approximately 10⁷, 10⁸, 10⁹, 10¹⁰, 10¹¹, 10¹², 10¹³, or even higher, infectious particles/mL, given either as a single dose (or divided into two or more administrations, etc.,) as may be required to achieve therapy of the particular disease or disorder being treated. In fact, in certain embodiments, it may be desirable to administer two or more different rAAV particle- or vector-based compositions, either alone, or in combination with one or more other diagnostic agents, drugs, bioactives, or such like, to achieve the desired effects of a particular regimen or therapy. In most rAAV-vectored, gene therapy-based regimens, the inventors contemplate that lower titers of infectious particles will be required when practicing the disclosed methods of pre-treating and co-administering AAV capsids with PVA.

To express a therapeutic agent in accordance with the present disclosure one may prepare a rAAV particle that comprises a therapeutic agent-encoding nucleic acid segment under the control of one or more promoters, e.g. a hepatic- or erythroid-tissue specific promoter. To bring a sequence “under the control of” a promoter, one positions the 5′ end of the transcription initiation site of the transcriptional reading frame generally between about 1 and about 50 nucleotides “downstream” of (i.e., 3′ of) the chosen promoter. The “upstream” promoter stimulates transcription of the DNA and promotes expression of the encoded polypeptide. This is the meaning of “recombinant expression” in this context. In some embodiments, recombinant vector constructs are those that include a capsid-protein modified rAAV vector that contains an RPE cell- or a photoreceptor cell-specific promoter, operably linked to at least one nucleic acid segment encoding one or more diagnostic, and/or therapeutic agents.

When the use of such vectors is contemplated for introduction of one or more exogenous proteins, polypeptides, peptides, ribozymes, and/or antisense oligonucleotides, to a particular cell transfected with the vector, one may employ the rAAV particles disclosed herein to deliver one or more exogenous polynucleotides to a selected host cell, e.g., to one or more selected cells within the mammalian eye.

In some embodiments, the number of viral particles administered to a subject may be on the order ranging from 10⁶ to 10¹⁴ particles/ml or 10³ to 10¹⁵ particles/ml, or any values therebetween for either range, such as for example, about 10⁶, 10⁷, 10⁸, 10⁹, 10¹⁰, 10¹¹, 10¹², 10¹³, or 10¹⁴ particles/ml. In one embodiment, viral particles of higher than 10¹³ particles/ml may be administered. In some embodiments, the number of viral particles administered to a subject may be on the order ranging from 10⁶ to 10¹⁴ vector genomes(vgs)/ml or 10³ to 10¹⁵ vgs/ml, or any values therebetween for either range, such as for example, about 10⁶, 10⁷, 10⁸, 10⁹, 10¹⁰, 10¹¹, 10¹², 10¹³, or 10¹⁴ vgs/ml. In one embodiment, viral particles of higher than 10¹³ vgs/ml are be administered. The viral particles can be administered as a single dose, or divided into two or more administrations as may be required to achieve therapy of the particular disease or disorder being treated. In some embodiments, 0.0001 ml to 10 ml, e.g., 0.001 ml, 0.01 ml, 0.1 ml, 1 ml, 2 ml, 5 ml or 10 ml, are delivered to a subject.

In some embodiments, the disclosure provides formulations of one or more viral-based compositions disclosed herein in pharmaceutically acceptable solutions for administration to a cell or an animal, either alone or in combination with one or more other modalities of therapy, and in particular, for therapy of human cells, tissues, and diseases affecting man.

If desired, rAAV particles described herein may be administered in combination with other agents as well, such as, e.g., proteins or polypeptides or various pharmaceutically-active agents, including one or more systemic or topical administrations of therapeutic polypeptides, biologically active fragments, or variants thereof. In fact, there is virtually no limit to other components that may also be included, given that the additional agents do not cause a significant adverse effect upon contact with the target cells or host tissues. The rAAV particles may thus be delivered along with various other agents as required in the particular instance. Such compositions may be purified from host cells or other biological sources, or alternatively may be chemically synthesized as described herein.

Formulation of pharmaceutically-acceptable buffer, excipients and carrier solutions is well-known to those of skill in the art, as is the development of suitable dosing and treatment regimens for using the particular compositions described herein in a variety of treatment regimens, including e.g., oral, parenteral, intravitreal, intraocular, intravenous, intranasal, intra-articular, and intramuscular administration and formulation.

Typically, these formulations may contain at least about 0.1% of the therapeutic agent (e.g., rAAV particle) or more, although the percentage of the active ingredient(s) may, of course, be varied and may conveniently be between about 1 or 2% and about 70% or 80% or more of the weight or volume of the total formulation. Naturally, the amount of therapeutic agent(s) in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

The term “excipient” refers to a diluent, adjuvant, carrier, or vehicle with which the rAAV particle is administered. Such pharmaceutical excipients can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers. Exemplary excipients and vehicles include, but are not limited to, HA, BSS, artificial CSF, PBS, Ringer's lactate solution, TMN200 solution, polysorbate 20, and poloxamer 100.

The amount of rAAV particle compositions and time of administration of such compositions will be within the purview of the skilled artisan having benefit of the present teachings. It is likely, however, that the administration of therapeutically-effective amounts of the disclosed compositions may be achieved by a single administration, such as for example, a single injection of sufficient numbers of viral particles to provide therapeutic benefit to the patient undergoing such treatment. Alternatively, in some circumstances, it may be desirable to provide multiple, or successive administrations of the compositions, either over a relatively short, or a relatively prolonged period of time, as may be determined by the medical practitioner overseeing the administration of such compositions.

The composition may include rAAV particles or nucleic acid vectors either alone, or in combination with one or more additional active ingredients, which may be obtained from natural or recombinant sources or chemically synthesized.

The rAAV particle, nucleic acid vector, and/or Rep protein (in any form contemplated herein) may be delivered in the form of a composition, such as a composition comprising the active ingredient, such as the rAAV particle, nucleic acid vector, and/or Rep protein (in any form contemplated herein), and a therapeutically or pharmaceutically acceptable carrier. The rAAV particles, Rep proteins, or nucleic acid vectors may be prepared in a variety of compositions, and may also be formulated in appropriate pharmaceutical vehicles for administration to human or animal subjects.

Other aspects of the disclosure are directed to methods that involve contacting cells with an rAAV preparation produced by a method described herein. The contacting may be, e.g., ex vivo or in vivo by administering the rAAV preparation to a subject. The rAAV particle or preparation may be delivered in the form of a composition, such as a composition comprising the active ingredient, such as a rAAV particle or preparation described herein, and a therapeutically or pharmaceutically acceptable excipient. The rAAV particles or preparations may be prepared in a variety of compositions, and may also be formulated in appropriate pharmaceutical vehicles for administration to human or animal subjects. In some embodiments, the nucleic acid vector comprises one or more regions comprising a sequence that facilitates expression of the nucleic acid (e.g., the transgene or the nucleic acid region encoding the Rep protein), e.g., expression control sequences operatively linked to the nucleic acid. Numerous such sequences are known in the art. Non-limiting examples of expression control sequences include promoters, insulators, silencers, response elements, introns, enhancers, initiation sites, termination signals, and poly(A) tails. Any combination of such control sequences is contemplated herein (e.g., a promoter and an enhancer).

To achieve appropriate expression levels of the protein or polypeptide of interest, any of a number of promoters suitable for use in the selected host cell may be employed. The promoter may be, for example, a constitutive promoter, tissue-specific promoter, inducible promoter, or a synthetic promoter. Inducible promoters and/or regulatory elements may also be contemplated for achieving appropriate expression levels of the protein or polypeptide of interest.

Tissue-specific promoters and/or regulatory elements are also contemplated herein. Exemplary tissue-specific promoters include mammalian liver tissue-specific promoters such as a transthyretin (TTR) promoter or an al anti-trypsin promoter. In other embodiments, the disclosed promoters are mammalian erthroid tissue-specific promoters such a β-globin promoter or a human parvovirus B19 promoter.

Synthetic promoters are also contemplated herein. A synthetic promoter may comprise, for example, regions of known promoters, regulatory elements, transcription factor binding sites, enhancer elements, repressor elements, and the like.

### Host Cells

The disclosure also contemplates host cells that comprise at least one of the disclosed rAAV particles or nucleic acid vectors described herein and optionally further comprise a Rep protein (e.g., in the form of a second rAAV particle, an mRNA, or the protein itself). Such host cells include mammalian host cells, with human host cells being preferred, and may be either isolated, in cell or tissue culture. In the case of genetically modified animal models (e.g., a mouse), the transformed host cells may be comprised within the body of a non-human animal itself.

The disclosure also contemplates host cells that comprise at least one of the disclosed rAAV particles or nucleic acid vectors. Such host cells include mammalian host cells, with human host cells being preferred, and may be either isolated, in cell or tissue culture. In the case of genetically modified animal models (e.g., a mouse), the transformed host cells may be comprised within the body of a non-human animal itself. In some embodiments, the host cell is a cancer cell. In some embodiments, the host cell is a liver cell, such as a liver cancer cell. In some embodiments, the host cell is a primary T cell, such as a primary human T cell. In some embodiments, the host cell is a bone marrow-derived CD34⁺ T cell.

In some embodiments, the host cells are human hematopoetic stem cells. In certain embodiments, the host cells are K562 cells.

In some embodiments, the host cells do not comprise bladder cells. In some embodiments, the host cells do not comprise bladder epithelial cells.

In some embodiments, a host cell as described herein is derived from a subject as described herein. Host cells may be derived using any method known in the art, e.g., by isolating cells from a fluid or tissue of the subject. In some embodiments, the host cells are cultured. Methods for isolating and culturing cells are well known in the art.

### Subjects

Aspects of the disclosure relate to methods and preparations for use with a subject, such as human or non-human primate subjects, a host cell in situ in a subject, or a host cell derived from a subject. Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. In some embodiments, the subject is a human subject. Other exemplary subjects include domesticated animals such as dogs and cats; livestock such as horses, cattle, pigs, sheep, goats, and chickens; and other animals such as mice, rats, guinea pigs, and hamsters.

In some embodiments, the subject has or is suspected of having a disease that may be treated with gene therapy. In some embodiments, the subject has or is suspected of having a hemoglobinopathy. A hemoglobinopathy is a disease characterized by one or more mutation(s) in the genome that results in abnormal structure of one or more of the globin chains of the hemoglobin molecule. Exemplary hemoglobinopathies include hemolytic anemia, sickle cell disease, and thalassemia. Sickle cell disease is characterized by the presence of abnormal, sickle-chalped hemoglobins, which can result in severe infections, severe pain, stroke, and an increased risk of death. Subjects having sickle cell disease can be identified, e.g., using one or more of a complete blood count, a blood film, hemoglobin electrophoresis, and genetic testing. Thalassemias are a group of autosomal recessive diseases characterized by a reduction in the amount of hemoglobin produced. Symptoms include iron overload, infection, bone deformities, enlarged spleen, and cardiac disease. The subgroups of thalassemias include alpha-thalassemia, beta-thalassemia, and delta thalassemia. Subjects having a thalassemia may be identified, e.g., using one or more of complete blood count, hemoglobin electrophoresis, Fe Binding Capacity, urine urobilin and urobilogen, peripheral blood smear, hematocrit, and genetic testing.

In some embodiments, the subject has or is suspected of having a disease that may be treated with gene therapy. In some embodiments, the subject has or is suspected of having a disease provided in Table 1.

In some embodiments, the subject has or is suspected of having a disease that may be treated with gene therapy. In some embodiments, the subject has or is suspected of having a proliferative disease, such as cancer. The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In some embodiments, the cancer is liver cancer. Exemplary liver cancers include, but are not limited to, hepatocellular carcinoma (HCC), cholangiocarcinoma, angiosarcoma, and hepatoblastoma. Subject having cancer can be identified by the skilled medical practitioner, e.g., using methods known in the art including biopsy, cytology, histology, endoscopy, X-ray, Magnetic Resonance Imaging (MRI), ultrasound, CAT scan (computerized axial tomography), genetic testing, and tests for detection of tumor antigens in the blood or urine.

### Intravenous Immunoglobulin (IVIG)

In some embodiments, the methods and compositions described herein are accompanied by administration of Intravenous immunoglobulin (IVIG). IVIG can provide the subject with a pool of antibodies to compensate for the loss of HLA-DR expression and/or antigen presentation. In some embodiments, IVIG is pooled, polyvalent, or IgG antibodies extracted from plasma healthy blood donors. Methods for producing IVIG are known in the art (see, e.g., Immune Deficiency Foundation. IDF Patient and Family Handbook: For Primary Immunodeficiency, Disease, 4th Ed. Towson, Md. 2007, and Jolles et al., Clin Exp Immunol. 2005 October; 142(1): 1-11). IVIG is also commercially available (see, e.g., GAMMAGARD LIQUID® and GAMMAGARD S/D® from Baxter Healthcare, GAMMAPLEX® from Bio Products Laboratory, FLEBOGAMMA® from Grifols, OCTAGAM® from Octapharma, PRIVIGEN® from CSL Behring, GAMUNEX® from Talecris Biotherapeutics, GAMMAKED® from Kedrion and BIVIGAM® from Biotest).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1—PVA Enhances the Transduction Efficiency of AAV6 Vectors in Human Hematopoietic Cells

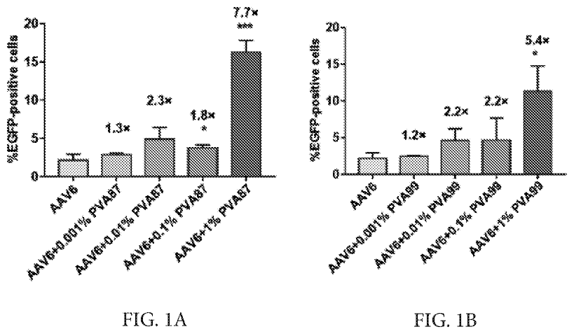

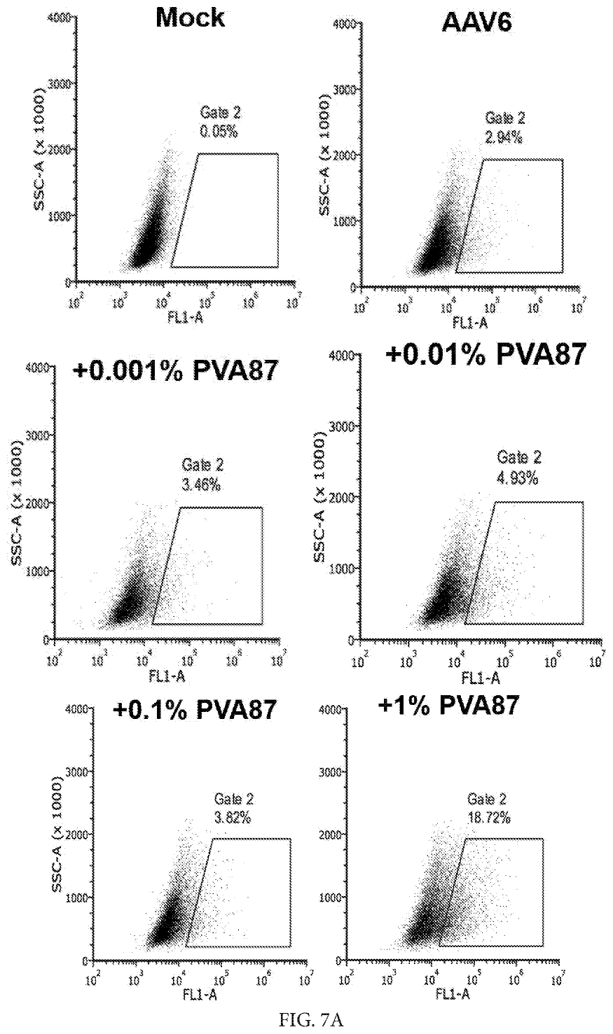

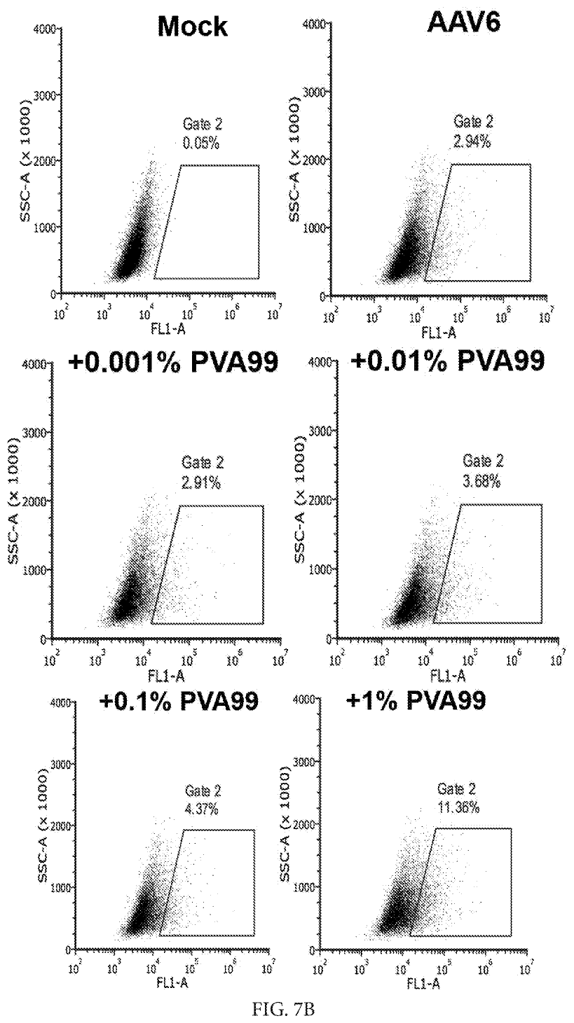

Since HSA has been shown to improve the transduction efficiency of AAV vectors, [39] and since PVA was recently shown to be a superior replacement for HSA to dramatically promote ex vivo expansion of murine HSCs, [37] the effect of PVA on the transduction efficiency of AAV6 vectors in primary human HSCs was evaluated. PVA is a synthetic polymer derived from polyvinyl acetate by hydrolysis. Typical levels of hydrolysis range from 80% to greater than 99%. [38] Both 87% hydrolyzed PVA (PVA87), and more than 99% hydrolyzed PVA (PVA99) was used in the preliminary experiments using K562 cells, frequently used as a model for hematopoietic cell transduction studies. AAV6 vectors expressing the enhanced green fluorescence protein (EGFP) reporter gene under the control of a cytomegalovirus (CMV) enhancer chicken b-actin promoter (CBA) were either mock-treated or pre-incubated with PVA concentration ranging from 0.001% to 1% and used to transduce K562 cells in triplicates under identical conditions. Transduction efficiency was evaluated by EGFP expression 48 h post-transduction using flow cytometry. These results are shown in FIGS. 1A-1B. As shown, whereas low concentration of PVA had no effect, a significant increase in the transduction efficiency of AAV6 vectors was observed with preincubation with 1% concentration of both PVA87 (FIG. 1A) and PVA99 (FIG. 1B). Since PVA is known to be non-cytotoxic, no apparent cytotoxicity in K562 cells was observed (FIGS. 7A and 7B). Since the extent of the increase in transgene expression with PVA87 was more pronounced than that with PVA99, all subsequent studies were carried out with PVA87.

### PVA-AAV6 Vector Interaction is Critical for the Increased Transduction Efficiency

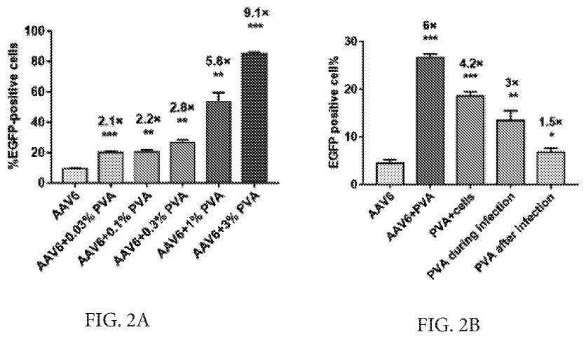

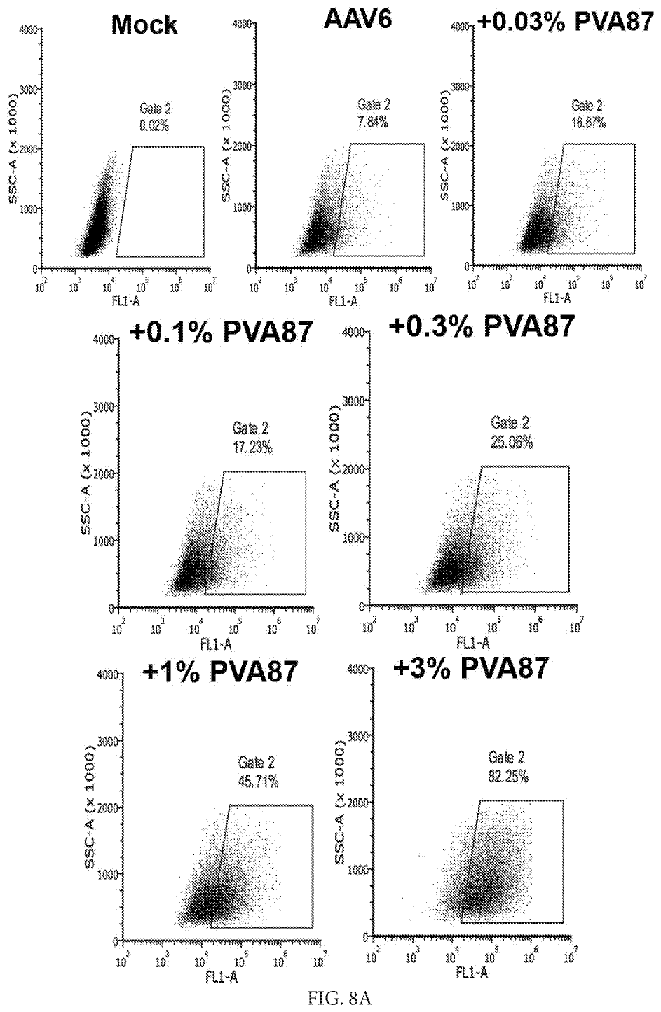

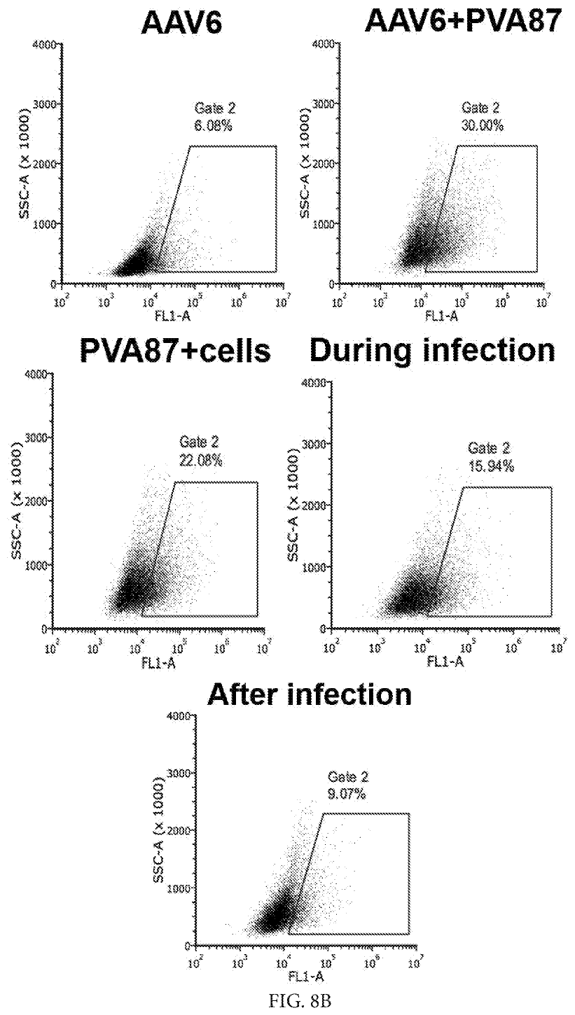

The transduction efficiency of AAV6 vectors was also evaluated to assess whether they could be further increased with higher concentration of PVA87. AAV6-CBA-EGFP vectors were preincubated with PVA87 at concentrations ranging from 0.03% to 3%, and transgene expression was determined as described above. These results, shown in FIG. 2A, document that a dose-dependent increase, up to 9-fold, in transgene expression was achieved with pre-incubation of AAV6 vectors with 3% PVA87. It was next of interest to determine which step in the AAV6 vector life cycle was influenced by PVA87. To this end, the following sets of experiments were performed: (1) no treatment; (2) pre-incubation of AAV6 vectors with 1% PVA87; (3) pre-incubation of K562 cells with 1% PVA87 followed by AAV6 vector transduction; (4) addition of 1% PVA87 prior to transduction with AAV6 vectors; and (5) addition of 1% PVA87 following transduction with AAV6 vectors. Transgene expression was determined as described above. These results are shown in FIG. 2B. It is evident that the extent of the increase in the transduction efficiency was most pronounced when PVA87 was pre-incubated with the vector, suggesting that a direct interaction between AAV6 and PVA87 is a critical step for the observed increase in the transduction efficiency, with no apparent cytotoxicity (FIGS. 8A-8B).

### PVA Enhances AAV6 Vector Uptake in Human Hematopoietic Cells

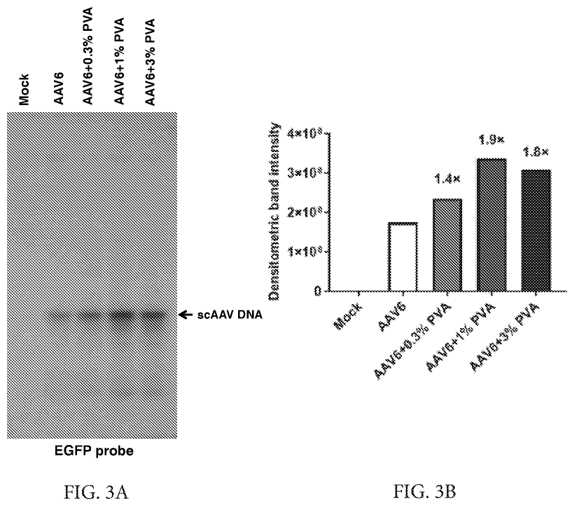

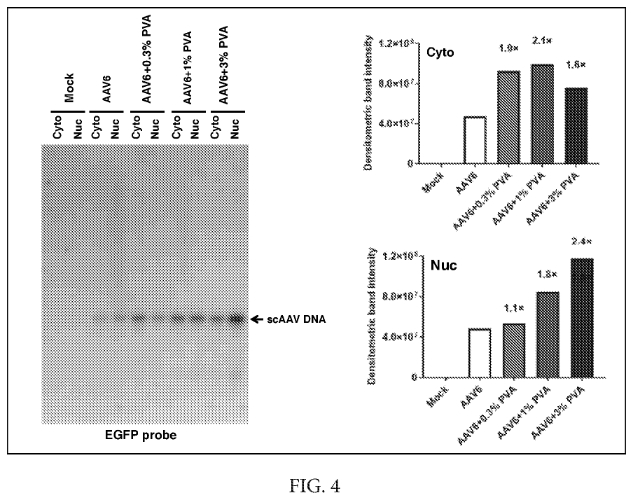

Since a direct interaction between AAV6 and PVA87 appeared to be critical, it was hypothesized that the observed increased transduction was a consequence of enhanced AAV6 vector uptake into cells. This possibility was tested experimentally by determining the extent of the vector entry in the absence or the presence of increasing amounts of PVA87, followed by detection of intracellular vector genome by Southern blot analysis. These results are shown in FIGS. 3A-3B. As can be seen, pre-incubation with PVA87 led to increased up-take of AAV vector genomes in K562 cells.

### PVA Enhances Nuclear Transport of AAV6 Vectors in Human Hematopoietic Cells

Since increased intracellular vector uptake alone could not account for increased transduction, unless the vectors also trafficked to the nucleus, the distribution of vector genomes in the cytoplasmic and the nuclear fractions was further analyzed. To this end, AAV6 vectors, with or without pre-incubation with increasing amounts of PVA87, were used to transduce K562 cells. Nuclear and cytoplasmic fractions were analyzed for the presence of vector genomes by Southern blot analysis as described above. These results, shown in FIG. 4, clearly indicate that pre-incubation with PVA87 also led to increased nuclear transport of AAV vector genomes into K562 cells and appeared to correlate well with the PVA87 concentration used.

### PVA Increases the Transduction Efficiency of AAV6 Vectors in Primary Human HSCs In Vitro

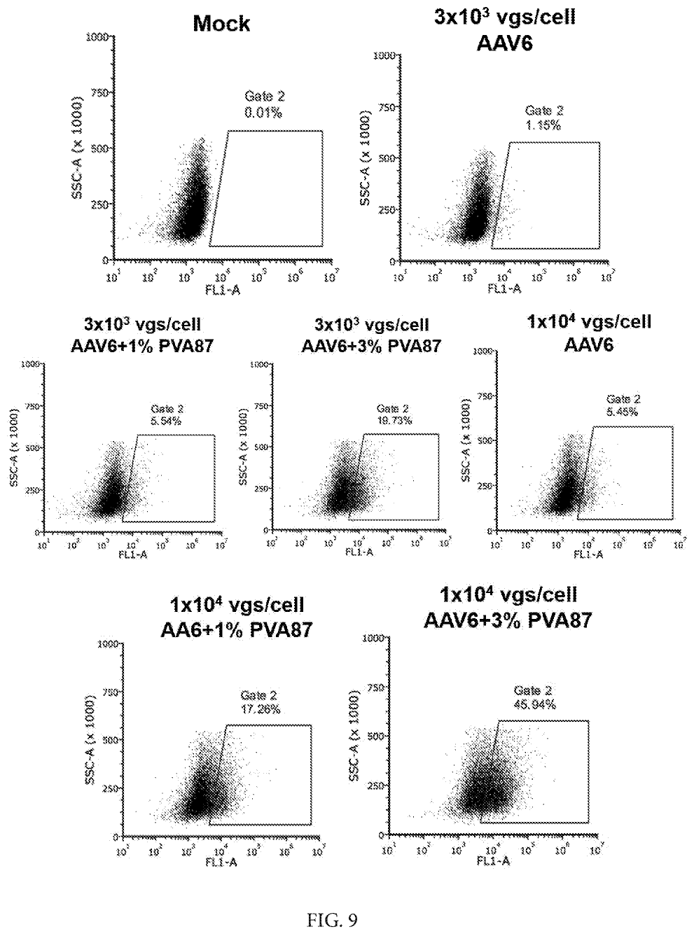

To further evaluate whether PVA87 could also increase the transduction efficiency of AAV6 vectors in HSCs, primary human bone marrow-derived CD34⁺ cells were transduced with self-complementary AAV6 (scAAV6)-CBAp-vectors at 3,000 and 10,000 vgs/cell, with or without pre-incubation with 1% or 3% PVA87, and analyzed them for transgene expression as described for K562 cells. These results are shown in FIG. 5. As can be seen, at 3,000 vgs/cell, up to 12-fold enhancement of transduction efficiency was observed with 3% PVA87, whereas the transduction efficiency was enhanced up to 8-fold with 3% PVA87 at 10,000 vgs/cell, with no apparent cytotoxicity (FIG. 9).

### Example 2—PVA Enhances the Transduction Efficiency of AAV6 Vectors in Murine Hepatocytes In Vivo with No Apparent Hepato-Toxicity

Whether PVA87 could augment the transduction efficiency of AAV6 vectors in an animal model in vivo was also examined. Because AAV6 vectors do not transduce primary murine HSCs. [21] the transduction efficiency of AAV6 vectors in mouse liver following intravenous delivery was evaluated. Three groups (n=5 each) of normal C57BL/6 mice were administered via the tail-vein with 1×10¹⁰ vgs of ssAAV6 vectors expressing the firefly luciferase (FLuc) reporter gene under the control of the CBA promoter, with or without pre-incubation with 1% or 3% PVA87. Two weeks post-vector administrations, whole-body bioluminescence imaging was performed and quantitated. These results are shown in FIG. 6A and FIG. 6B. It is interesting to note that pre-incubation with 1% PVA87 led to 4-fold increase, and with 3% PVA87 led to 7-fold increase in AAV6 transduction efficiency in murine hepato-cytes in vivo. These results suggested that the observed PVA87-mediated increased efficiency was also due to increased intracellular trafficking of AAV6 vectors in murine hepatocytes. Vector genome copy numbers were determined in DNA samples from liver tissues from mice from each of the three groups using quantitative polymerase chain reaction (qPCR) with FLuc-specific primers. These results, shown in FIG. 6C, corroborated that the PVA87-mediated improved transduction efficiency correlated with increased vector uptake and entry into mouse hepatocytes in vivo. The potential PVA-induced liver toxicity was examined by determining the blood levels of aspartate aminotrans-ferase (AST) and alanine aminotransferase (ALT), which are indicative of liver damage. 2 weeks post-vector administration, both AST and ALT levels in mice from each of the three groups were observed to be within the normal range (data not shown), indicating that PVA-treatment is not associated with liver toxicity at the concentration tested.

### Discussion

Since the first identification of AAV6 as the most efficient serotype vector for transduction of primary human HSCs, [21] several other independent groups have not only corroborated these observations, but further documented that AAV6 vectors are also highly efficient in mediating genome editing in primary human HSCs, as well as in primary human T cells.[22-24] However, relatively high doses, ranging from 100,000-200,000 vgs/cell are required to achieve 25%-55% efficacy, including genome editing of the sickle mutation with the CRISPR/Cas9 system. [25] In order to reduce, if not completely eliminate, the possibility of off-target cleavage, it would be desirable to limit the vector dose to as low a level as possible.

The development of capsid-modified AAV6 vectors with which transduction efficiency exceeding 90% can be achieved has been reported in primary human HSCs at an MOI of 20,000 vgs/cell. [39], [40] Thus, it is conceivable that PVA could be safely used to further reduce the AAV6 vector dose to achieve safe and efficient genome editing in HSCs from patients with b-thalassemia and sickle cell disease, especially since PVA has been widely used in a number of medical devices, due to its biocompatibility, low toxicity, and low protein adsorption characteristics. [38] Additionally, PVA was not found to induce liver toxicity in mice. In this context, however, it is also important to note that although AAV6 vectors efficiently deliver donor DNA templates in conjunction with nuclease-based editing platforms such as zinc finger nucleases and CRISPR/Cas9, [22-25] the AAV6-delivered donors are recombined at the sites of double-stranded DNA breaks induced by nucleases such as CRISPR, and that AAV6 alone does not induce genome editing in the absence of nuclease treatment. [41-43]

More recently, novel human stem-cell-derived clade F AAV vectors were shown to mediate high-efficiency homologous recombination based genome editing in primary human HSCs. [44] Although it has been claimed that AAV6 is superior to clade F AAV vectors in mediating homologous recombination-based genome editing in human HSCs, [45] and that homologous recombination-based genome editing by clade F AAV vectors is inefficient in the absence of a targeted DNA break, [46] the underlying reason for these apparent discrepancies has been explained by the requirement of exceedingly high MOIs, 150,000 vgs/cell or higher, to achieve nuclease-free homologous recombination by clade F AAV vectors. [47] Thus, it would be of interest to evaluate whether PVA-treatment can augment the genome editing efficiency of AAVHSC vectors as well, thereby significantly reducing the need to use high MOIs.

In the experiments disclosed herein, it was observed that PVA, which was recently reported to be a superior replacement for HSA in a dramatic ex vivo expansion of murine HSCs, [37] could also significantly improve the transduction efficiency of AAV6 vectors in primary human HSCs. In this context, it is noteworthy that significant expansion of primary human HSCs in a zwitterionic hydrogel in a 3D culture was recently reported. [48] What effect, if any, these conditions have on the transduction efficiency of AAV6 vectors remains to be investigated. Additional studies are ongoing to gain a better understanding of the underlying mechanism of AAV6-PVA interactions. It was demonstrated that the improvement in the transduction efficiency was due to PVA mediated improved entry and intracellular trafficking of AAV6 vectors in human hematopoietic cells in vitro, as well as in murine hepatocytes in vivo. The PVA-mediated enhancement of transduction reported herein may be specific to certain clinically relevant AAV serotypes, such as AAV6 and AAV3. Studies are ongoing to determine the transduction efficiency enhancement effects of PVA on AAVrh.74, AAV5, AAV8, and AAV9. In this context, however, it is noteworthy that PVA did not appreciably augment the transduction efficiency of wild-type AAV2 serotype vectors in several human cell lines in vitro (H. Y., K. Q., M. T., W. W., A. S., unpublished data). This disparity in transduction behavior may be due to differences in relevant amino acid residues in the AAV6 and AAV2 serotypes that are exposed to the milieu. At the same time, it is appreciated that PVA may appreciably augment the transduction efficiency of one or more AAV2 capsid variant vectors in human cell lines.

### Materials and Methods

### Cell Lines, Primary Cells, Cell Cultures, and Reagents

Human embryonic kidney 293 (HEK293) and erythroleukemia K562 cells were purchased from American Type Culture Collections (ATCC, Manassas, Va., USA) and maintained at 37° C. in 5% CO₂ in Dulbecco's modified Eagle's medium (DMEM; Lonza, Walkersville, Md., USA) supplemented with 10% fetal bovine serum (FBS; Sigma, St. Louis, Mo., USA) and 1% penicillin-streptomycin (Invitrogen, Grand Island, N.Y., USA). Human bone marrow CD34⁺ cells were purchased from AllCells (AllCells Technologies, Emeryville, Calif., USA) and maintained at 37° C. in 5% CO₂ in Stem-Span Serum-Free Expansion Medium (SFEM; StemCell Technologies, Vancouver, BC, Canada) with StemSpan CC100 (StemCell Technologies, Vancouver, BC, Canada). PVA87 (87%-90% hydrolyzed, average molecular weight 30,000-70,000; catalog no. P8136) and PVA99 (99+% hydrolyzed, average molecular weight 85,000-124,000; catalog no. 363146) were purchased from Sigma-Aldrich, St. Louis, Mo., USA. PVA87 (87%-90% hydrolyzed, average molecular weight 30,000-70,000; catalog no. P8136) and PVA99 (99+% hydrolyzed, average molecular weight 85,000-124,000; catalog no. 363146) were purchased from Sigma-Aldrich, St. Louis, Mo., USA.

### Viral Vector Production

The scAAV plasmid containing the CBAp-EGFP transgene expression cassette has been described previously. [49] scAAV6-CBAp-EGFP vector was packaged using the triple-plasmid transfection method, mediated by polyethyleneimine [50] (PEI, linear, MW 25000; Polysciences, Warrington, Pa., USA). HEK293 cells were harvested 72 h post-transfection, and lysed by 3 rounds of freezethaw, and digested with Benzonase (Invitrogen, Grand Island, N.Y., USA). Cell debris was removed by centrifugation. AAV6 vectors were purified by iodixanol (Sigma, St. Louis, Mo., USA) gradient ultracentrifugation, followed by ion exchange chromatography using HiTrap SP/Q HP columns (GE Healthcare, Piscataway, N.J., USA), washed with PBS and concentrated by centrifugation using centrifugal spin concentrators with 150 kDa molecular weight cutoff. Titers were determined by quantitative real-time PCR assays as previously described. rAAV6-CBAp-FLuc was purchased from PackGene (PackGene Biotech, Worcester, Mass., USA).

### AAV Transduction Assay In Vitro Human

K562 cells (1×10⁵) and primary human CD34⁺ cells (5×10⁴) were seeded in Falcon round bottom polystyrene tubes (Corning, N.Y., USA). Cells were resuspended in DMEM before transduction. scAAV6 vectors expressing the EGFP reporter gene under the control of a chimeric cytomegalovirus (CMV) enhancer/chicken beta-actin promoter (CBA) were either mock-treated or pre-incubated with PVA concentration ranging from 0.001% to 3% and used to transduce cells in triplicates under identical conditions. DMEM was replaced by culture medium 2 h post-transduction. EGFP expression was determined 48 h post-transduction using flow cytometry (Accuri C6, Beckton Dickinson, Franklin Lakes, N.J., USA), followed by processing with software FCS Express 6 Flow.

### Southern Blot Analyses

Low molecular weight DNA samples isolated from whole-cells or nuclear and cytoplasmic fractions from K562 cells were isolated by the method described by Hirt. [51] DNA fragments were electrophoresed on 1% agarose gels, transferred to nylon membranes, hybridized with 32P-labeled EGFP DNA probe and subjected to autoradiography. Densitometric scanning of autoradiograms was performed with a Digital Imaging System Alphaimager (Alpha Innotech, San Leandro, Calif., USA).

### Animal Experiments

Male C57BL/6 mice were purchased from the Jackson Laboratory and maintained in the University of Florida Animal Care Facility. All experimental protocols involving animals were approved by the Institutional Animal Care and Use Committee guidelines. 1×10¹⁰ particles of ssAAV6-CBAp-FLuc vectors were incubated with or without PVA, in a total volume of 200 mL, at 4° C. for 2 h, followed by tail-vein injections (n=5 per group). 2 weeks post-vector injections, whole-body bioluminescence imaging was performed using a Xenogen IVIS Lumina imaging system (Caliper Lifesciences, Hopkinton, Mass., USA) following intraperitoneal injection of luciferin substrate (Nanolight, Pinetop, Ariz., USA) at 150 mg per kg of mouse body weight. Bioluminescence images were analyzed using Living Image software (Caliper Lifesciences, Hopkinton, Mass., USA). AAV6 vector genome copy numbers were determined by qPCR using total genomic DNA isolated from liver tissues.

### Statistical Analysis

Quantitative results were expressed as means±SD. Unpaired Student's t test was performed for statistical analysis. p values <0.05 were considered as statistically significant. Statistical significances are indicated as *p<0.05, **p<0.01, ***p<0.001.

### REFERENCES

The following references, to the extent that they provide exemplary procedural or other details supplementary to those set forth herein, are specifically incorporated herein by reference:
1. Dzierzak E A, Papayannopoulou T, Mulligan R C. Lineage-specific expression of a human beta-globin gene in murine bone marrow transplant recipients reconstituted with retrovirus-transduced stem cells. Nature 331, 35-41 (1988).
2. May C, et al. Therapeutic haemoglobin synthesis in beta-thalassaemic mice expressing lentivirus-encoded human beta-globin. Nature 406, 82-86 (2000).
3. Pawliuk R, et al. Correction of sickle cell disease in transgenic mouse models by gene therapy. Science 294, 2368-2371 (2001).
4. Imren S, et al. Permanent and panerythroid correction of murine beta thalassemia by multiple lentiviral integration in hematopoietic stem cells. Proc Natl Acad Sci USA 99, 14380-14385 (2002).
5. Persons D A, et al. Successful treatment of murine beta-thalassemia using in vivo selection of genetically modified, drug-resistant hematopoietic stem cells. Blood 102, 506-513 (2003).
6. Rivella S, May C, Chadburn A, Riviere I, Sadelain M. A novel murine model of Cooley anemia and its rescue by lentiviral-mediated human beta-globin gene transfer. Blood 101, 2932-2939 (2003).
7. Imren S, et al. High-level beta-globin expression and preferred intragenic integration after lentiviral transduction of human cord blood stem cells. J Clin Invest 114, 953-962 (2004).
8. Puthenveetil G, et al. Successful correction of the human beta-thalassemia major phenotype using a lentiviral vector. Blood 104, 3445-3453 (2004).
9. Cavazzana-Calvo M, et al. Transfusion independence and HMGA2 activation after gene therapy of human beta-thalassaemia. Nature 467, 318-322 (2010).
10. Ribeil J A, et al. Gene Therapy in a Patient with Sickle Cell Disease. N Engl J Med 376, 848-855 (2017).
11. Thompson A A, et al. Gene Therapy in Patients with Transfusion-Dependent beta-Thalassemia. N Engl J Med 378, 1479-1493 (2018).
12. Hargrove P W, et al. Globin lentiviral vector insertions can perturb the expression of endogenous genes in beta-thalassemic hematopoietic cells. Mol Ther 16, 525-533 (2008).
13. Arumugam P I, et al. Genotoxic potential of lineage-specific lentivirus vectors carrying the beta-globin locus control region. Mol Ther 17, 1929-1937 (2009).
14. Bokhoven M, et al. Insertional gene activation by lentiviral and gammaretroviral vectors. J Virol 83, 283-294 (2009).
15. Heckl D, et al. Lentiviral vector induced insertional haploinsufficiency of Ebf1 causes murine leukemia. Mol Ther 20, 1187-1195 (2012).
16. Tan M, Qing K, Zhou S, Yoder M C, Srivastava A. Adeno-associated virus 2-mediated transduction and erythroid lineage-restricted long-term expression of the human betaglobin gene in hematopoietic cells from homozygous beta-thalassemic mice. Mol Ther 3, 940-946 (2001).
17. Ponnazhagan S, et al. Adeno-associated virus type 2-mediated transduction in primary human bone marrow-derived CD34⁺ hematopoietic progenitor cells: donor variation and correlation of transgene expression with cellular differentiation. J Virol 71, 8262-8267 (1997).
18. Han Z, et al. Stable integration of recombinant adeno-associated virus vector genomes after transduction of murine hematopoietic stem cells. Hum Gene Ther 19, 267-278 (2008).
19. Maina N, et al. Recombinant self-complementary adeno-associated virus serotype vector-mediated hematopoietic stem cell transduction and lineage-restricted, long-term transgene expression in a murine serial bone marrow transplantation model. Hum Gene Ther 19, 376-383 (2008).
20. Maina N, et al. Optimization of recombinant adeno-associated viral vectors for human beta-globin gene transfer and transgene expression. Hum Gene Ther 19, 365-375 (2008).
21. Song L, et al. Optimizing the transduction efficiency of capsid-modified AAV6 serotype vectors in primary human hematopoietic stem cells in vitro and in a xenograft mouse model in vivo. Cytotherapy 15, 986-998 (2013).
22. Sather B D, et al. Efficient modification of CCR5 in primary human hematopoietic cells using a megaTAL nuclease and AAV donor template. Sci Transl Med 7, 307ra156 (2015).
23. Wang J, et al. Homology-driven genome editing in hematopoietic stem and progenitor cells using ZFN mRNA and AAV6 donors. Nat Biotechnol 33, 1256-1263 (2015).
24. De Ravin S S, et al. Targeted gene addition in human CD34(+) hematopoietic cells for correction of X-linked chronic granulomatous disease. Nat Biotechnol 34, 424-429 (2016).
25. Dever D P, et al. CRISPR/Cas9 beta-globin gene targeting in human haematopoietic stem cells. Nature 539, 384-389 (2016).
26. McCarty D M, Monahan P E, Samulski R J. Self-complementary recombinant adeno-associated virus (scAAV) vectors promote efficient transduction independently of DNA synthesis. Gene Ther 8, 1248-1254 (2001).
27. McCarty D M, Fu H, Monahan P E, Toulson C E, Naik P, Samulski R J. Adeno-associated virus terminal repeat (TR) mutant generates self-complementary vectors to overcome the rate-limiting step to transduction in vivo. Gene Ther 10, 2112-2118 (2003).
28. Wu Z, et al. Optimization of self-complementary AAV vectors for liver-directed expression results in sustained correction of hemophilia B at low vector dose. Mol Ther 16, 280-289 (2008).
29. Ponnazhagan S, Woody M J, Wang X S, Zhou S Z, Srivastava A. Transcriptional transactivation of parvovirus B19 promoters in nonpermissive human cells by adenovirus type 2. J Virol 69, 8096-8101 (1995).
30. Wang X S, Yoder M C, Zhou S Z, Srivastava A. Parvovirus B19 promoter at map unit 6 confers autonomous replication competence and erythroid specificity to adenoassociated virus 2 in primary human hematopoietic progenitor cells. Proc Natl Acad Sci USA 92, 12416-12420 (1995).
31. Zhong L, et al. Next generation of adeno-associated virus 2 vectors: point mutations in tyrosines lead to high-efficiency transduction at lower doses. Proc Natl Acad Sci USA 105,
32. Kauss M A, Smith L J, Zhong L, Srivastava A, Wong K K, Jr., Chatterjee S. Enhanced long-term transduction and multilineage engraftment of human hematopoietic stem cells transduced with tyrosine-modified recombinant adeno-associated virus serotype 2. Hum Gene Ther 21, 1129-1136 (2010).
33. Ussher J E, Taylor J A. Optimized transduction of human monocyte-derived dendritic cells by recombinant adeno-associated virus serotype 6. Hum Gene Ther 21, 1675-1686 (2010).
34. Cheng B, et al. Development of optimized AAV3 serotype vectors: mechanism of high-efficiency transduction of human liver cancer cells. Gene Ther 19, 375-384 (2012).
35. Dalkara D, Byrne L C, Lee T, Hoffmann N V, Schaffer D V, Flannery J G. Enhanced gene delivery to the neonatal retina through systemic administration of tyrosine-mutated AAV9. Gene Ther 19, 176-181 (2012).
36. Wang M, et al. Direct interaction of human serum proteins with AAV virions to enhance AAV transduction: immediate impact on clinical applications. Gene Ther 24, 49-59 (2017).
37. Wilkinson A C, et al. Long-term ex vivo haematopoietic-stem-cell expansion allows nonconditioned transplantation. Nature 571, 117-121 (2019).
38. Baker, M. I., Walsh, S. P., Schwartz, Z., and Boyan, B. D. (2012). A review of polyvinyl alcohol and its uses in cartilage and orthopedic applications. J. Biomed. Mater. Res. B Appl. Biomater. 100, 1451-1457.
39. Song, L., Li, X., Jayandharan, G. R., Wang, Y., Aslanidi, G. V., Ling, C., Zhong, L., Gao, G., Yoder, M. C., Ling, C., et al. (2013). High-efficiency transduction of primary human hematopoietic stem cells and erythroid lineage-restricted expression by optimized AAV6 serotype vectors in vitro and in a murine xenograft model in vivo. PLoS ONE 8, e58757.
40. Ling, C., Bhukhai, K., Yin, Z., Tan, M., Yoder, M. C., Leboulch, P., Payen, E., and Srivastava, A. (2016). High-Efficiency Transduction of Primary Human Hematopoietic Stem/Progenitor Cells by AAV6 Vectors: Strategies for Overcoming Donor-Variation and Implications in Genome Editing. Sci. Rep. 6, 35495.
41. Bak, R. O., Dever, D. P., Reinisch, A., Cruz Hernandez, D., Majeti, R., and Porteus, M. H. (2017). Multiplexed genetic engineering of human hematopoietic stem and progenitor cells using CRISPR/Cas9 and AAV6. eLife 6, e27873.
42. Cromer, M. K., Vaidyanathan, S., Ryan, D. E., Curry, B., Lucas, A. B., Camarena, J., Kaushik, M., Hay, S. R., Martin, R. M., Steinfeld, I., et al. (2018). Global transcriptional response to CRISPR/Cas9-AAV6-based genome editing in CD34+ hematopoietic stem and progenitor cells. Mol. Ther. 26, 2431-2442.
43. Martin, R. M., Ikeda, K., Cromer, M. K., Uchida, N., Nishimura, T., Romano, R., Tong, A. J., Lemgart, V. T., Camarena, J., Pavel-Dinu, M., et al. (2019). Highly efficient and marker-free genome editing of human pluripotent stem cells by CRISPR-Cas9 RNP and AAV6 donor-mediated homologous recombination. Cell Stem Cell 24, 821-828.e5.
44. Smith, L. J., Wright, J., Clark, G., Ul-Hasan, T., Jin, X., Fong, A., Chandra, M., St Martin, T., Rubin, H., Knowlton, D., et al. (2018). Stem cell-derived clade F AAVs mediate high-efficiency homologous recombination-based genome editing. Proc. Natl. Acad. Sci. USA 115, E7379-E7388.
45. Dudek, A. M., and Porteus, M. H. (2019). AAV6 Is Superior to Clade F AAVs in Stimulating Homologous Recombination-Based Genome Editing in Human HSPCs. Mol. Ther. 27, 1701-1705.
46. Rogers, G. L., Chen, H.-Y., Morales, H., and Cannon, P. M. (2019). Homologous recombination-based genome editing by clade F AAVs is inefficient in the absence of a targeted DNA break. Mol. Ther. 27, 1726-1736.
47. Chatterjee, S. (2019). Efficient nuclease-free HR by clade F AAV requires high MOIs with high quality vectors. Mol. Ther. 27, 2058-2061.
48. Bai, T., Li, J., Sinclair, A., Imren, S., Merriam, F., Sun, F., O'Kelly, M. B., Nourigat, C., Jain, P., Delrow, J. J., et al. (2019). Expansion of primitive human hematopoietic stem cells by culture in a zwitterionic hydrogel. Nat. Med. 25, 1566-1575.
49. Ma, W., Li, B., Ling, C., Jayandharan, G. R., Srivastava, A., and Byrne, B. J. (2011). A simple method to increase the transduction efficiency of single-stranded adeno-associated virus vectors in vitro and in vivo. Hum. Gene Ther. 22, 633-640.
50. Dong, B., Moore, A. R., Dai, J., Roberts, S., Chu, K., Kapranov, P., Moss, B., and Xiao, W. (2013). A concept of eliminating nonhomologous recombination for scalable and safe AAV vector generation for human gene therapy. Nucleic Acids Res. 4141, 6609-6617.
51. Hirt, B. (1967). Selective extraction of polyoma DNA from infected mouse cell cultures. J. Mol. Biol. 26, 365-369.

### EQUIVALENTS

It should be understood that the examples and embodiments described herein are for illustrative purposes only and that various modifications or changes in light thereof will be suggested to persons skilled in the art and are to be included within the spirit and purview of this application and the scope of the appended claims. All references, including publications, patent applications and patents, cited herein are hereby incorporated by reference to the same extent as if each reference was individually and specifically indicated to be incorporated by reference and was set forth in its entirety herein. Recitation of ranges of values herein are merely intended to serve as a shorthand method of referring individually to each separate value falling within the range, unless otherwise indicated herein, and each separate value is incorporated into the specification as if it were individually recited herein.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are chemically and/or physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method comprising co-administering an rAAV particle with polyvinyl alcohol (PVA) to a mammal, wherein the rAAV particle comprises a capsid of a serotype of AAV6, AAV3, or a variant thereof.

2. The method of claim 1, wherein the serotype of the capsid is AAV3.

3. The method of claim 1, wherein the serotype of the capsid is AAV6.

4. The method of any one of claims 1-3, wherein the capsid of the rAAV particle is pre-incubated with PVA prior to administration to the mammal.

5. The method of any one of claims 1-4, wherein the capsid is pre-incubated with a buffer that comprises PVA.

6. The method of claim 4, wherein the capsid is pre-incubated with the PVA for a duration of about 5 minutes, about 15 minutes, about 30 minutes, about 45 minutes, about 60 minutes, about 75 minutes, about 90 minutes, or about 180 minutes.

7. The method of claim 6, wherein the capsid is pre-incubated for a duration of about 15 minutes.

8. The method of any one of claims 4-7, wherein the capsid is pre-incubated with PVA in a concentration of 0.01%, 0.05%, 0.1%, 0.2%, 0.3%, 0.4%, 0.5%, 0.6%, 0.75%, 0.9%, 1.0%, 1.25%, 1.5%, 1.75%, 2.0%, 2.25%, 2.5%, 2.75%, 3.0%, 3.25%, 3.5%, 3.75%, 4.0%, 5.0%, or 6.0% weight by volume.

9. The method of any one of claims 1-8, wherein the rAAV particle is administered in a titer of about 1×10⁶ vg/ml 1×10⁷ vg/ml, 1×10⁸ vg/ml, 5×10⁸ vg/ml, 1×10⁹ vg/ml, 5×10⁹ vg/ml, 1×10¹⁰ vg/ml, 5×10¹⁰ vg/ml, 1×10¹¹ vg/ml, 5×10¹¹ vg/ml, 1×10¹² vg/ml, 2×10¹² vg/ml, 3×10¹² vg/ml, 4×10¹² vg/ml, or 5×10¹² vg/ml.

10. The method of any one of claims 1-9, wherein the rAAV particle is administered in a titer of less than 1×10⁹ vg/ml.

11. The method of any one of claims 1-10, wherein administration is provided by an intravenous injection in a volume of about 500 μL, about 300 μL, about 250 μL, about 200 μL, about 175 μL, about 160 μL, about 145 μL, about 130 μL, about 115 μL, about 100 μL, about 90 μL, about 80 μL, about 70 μL, about 60 μL, about 55 μL, about 50 μL, about 45 μL, about 35 μL, about 20 μL, about 10 μL, or about 5 μL.

12. The method of any one of claims 4-11, wherein the injection is provided in a volume of about 50 μL.

13. The method of any one of claims 1-12, wherein the PVA is 87% hydrolyzed.

14. The method of any one of claims 1-13, wherein the rAAV particle further comprises a polynucleotide comprising a heterologous nucleic acid sequence.

15. The method of claim 14, wherein the heterologous nucleic acid sequence is operably linked to a promoter that directs expression of the heterologous nucleic acid sequence in a hepatic cell.

16. The method of claim 15, wherein the promoter comprises a CBA promoter, a β-globin promoter, a human parvovirus B19 promoter, a transthyretin (TTR) promoter, or an al anti-trypsin promoter.

17. The method of claim 15 or 16, wherein the promoter comprises a β-globin promoter or a human parvovirus B19 promoter.

18. The method of any one of claims 1-17, wherein the heterologous nucleic acid sequence encodes a therapeutic agent.

19. The method of claim 18, wherein the therapeutic agent is selected from human β-globin and human γ-globin.

20. The method of claim 18, wherein the therapeutic agent is selected from clotting Factor IX (FIX) and Factor VIII (FVIII).

19. A method for providing a mammal in need thereof with a therapeutically effective amount of the therapeutic agent of claim 17 or 18, the method comprising co-administering an rAAV particle with polyvinyl alcohol to a mammal for a time effective to provide the mammal with a therapeutically-effective amount of the therapeutic agent, wherein the rAAV particle comprises a capsid of an AAV6 or an AAV3 serotype, or a variant thereof.

20. A method for treating or ameliorating one or more symptoms of a disease, disorder or condition, the method comprising co-administering an rAAV particle with polyvinyl alcohol to a mammal in need thereof for a time sufficient to treat or ameliorate the one or more symptoms of the disease, disorder or condition in the mammal, wherein the rAAV particle comprises i) a polynucleotide encoding a therapeutic agent and ii) an AAV6 or an AAV3 capsid, or a variant thereof.

21. The method of claim 19 or 20, wherein the capsid is an AAV6 capsid.

22. The method of claim 19 or 20, wherein the capsid is selected from an AAV3, AAV3b, and AAVLK03 capsid.

23. The method of claim 19 or 20, wherein the capsid comprises non-native amino acid substitutions at amino acid residues of a wild-type AAV6 capsid, wherein the non-native amino acid substitutions comprise one or more of Y445F, Y705F, Y731F, T492V and S663V.

24. The method of claim 19 or 20, wherein the capsid comprises non-native amino acid substitutions of a wild-type AAV6 capsid, wherein the non-native amino acid substitutions comprise:
(a) Y445F;
(b) Y705F+Y731F;
(c) T492V;
(d) Y705F+Y731F+T492V;
(e) S663V; or
(f) S663V+T492V.

25. A method for treating or ameliorating one or more symptoms of a disease, disorder or condition, the method comprising co-administering an rAAV particle with polyvinyl alcohol to a mammal in need thereof for a time sufficient to treat or ameliorate the one or more symptoms of the disease, disorder or condition in the mammal, wherein the rAAV particle comprises i) a polynucleotide encoding a therapeutic agent and ii) an AAV5, an AAV8 or an AAV9 capsid, or a variant thereof.

26. The method of claim 25 wherein the capsid comprises non-native amino acid substitutions at amino acid residues 533 and/or 733 of a wild-type AAV8 capsid, wherein the non-native amino acid substitution is E533K and/or Y733F.

27. The method of any one of claims 19-26, wherein the mammal is human.

28. The method of any one of claims 19-27, wherein the disease, disorder or condition is β-thalassemia or sickle cell disease.

29. The method of any one of claims 19-28, wherein production of the therapeutic agent preserves one or more hepatic cells or hematopoetic stem cells.

30. A buffer for storing a mixture of AAV and polyvinyl alcohol (PVA), comprising:
(a) PVA in a concentration of 0.01%, 0.05%, 0.1%, 0.2%, 0.3%, 0.4%, 0.5%, 0.6%, 0.75%, 0.9%, 1.0%, 1.25%, 1.5%, 1.75%, 2.0%, 2.25%, 2.5%, 2.75%, 3.0%, 3.25%, 3.5%, 3.75%, 4.0%, 5.0%, or 6.0% weight by volume;
(b) balanced salt solution (BSS);
(c) artificial cerebrospinal fluid; and/or
(d) phosphate buffered saline (PBS).

31. The buffer of claim 41 further comprising:
(e) Ringer's lactate solution; and
(f) TMN200 solution.

32. The buffer of claim 30 or 31, wherein the PVA is in a concentration of 1.0% or 3.0% weight by volume.

33. A method comprising co-administering an rAAV particle with polyvinyl alcohol (PVA) to a mammal, wherein the rAAV particle comprises a capsid of a serotype of AAVrh.74, or a variant thereof.

34. A method for providing a mammal in need thereof with a therapeutically effective amount of the therapeutic agent of claim 17 or 18, the method comprising co-administering an rAAV particle with polyvinyl alcohol to a mammal for a time effective to provide the mammal with a therapeutically-effective amount of the therapeutic agent, wherein the rAAV particle comprises a capsid of an AAVrh.74 serotype.

35. A method for treating or ameliorating one or more symptoms of a disease, disorder or condition, the method comprising co-administering an rAAV particle with polyvinyl alcohol to a mammal in need thereof for a time sufficient to treat or ameliorate the one or more symptoms of the disease, disorder or condition in the mammal, wherein the rAAV particle comprises i) a polynucleotide encoding a therapeutic agent and ii) an AAVrh.74 capsid.
